(19) United States
(12) Patent Application Publication
(10) Pub. No.: US 2006/0047261 A1
(43) Pub. Date: Mar. 2, 2006

(54) Intra-arterial catheter for drug delivery

(76) Inventor: Shailendra Joshi, Edgewater, NJ (US)

Correspondence Address: BROWN RAYSMAN MILLSTEIN FELDER & STEINER LLP, 900 THIRD AVENUE, NEW YORK, NY, 10022, US

(21) Appl. No.: 10/879,850

(22) Filed: Jun. 28, 2004

(51) Int. Cl. A61M 31/00

(52) U.S. Cl. 604/509; 604/96.01

(57) ABSTRACT

The present invention provides a catheter, a drug delivery system and methods for the localized delivery of therapeutic or diagnostic agent to a target location in a subject and methods for the treatment of a pathological disorder in a subject using the same.

### STATEMENT OF GOVERNMENT INTEREST

This invention was made with PARTIAL government support under NIH Grant No. GM K08 000698. As such, the United States government has certain rights in this invention.

### FIELD OF THE INVENTION

The invention disclosed herein generally relates to medical devices for localized drug delivery and uses thereof.

### BACKGROUND OF THE INVENTION

A plurality of methods have been developed for the delivery of a pharmaceutical composition to treat various medical conditions. A pharmaceutical composition may be provided to a subject, e.g., a human or a veterinary patient, in need of therapeutic treatment via a variety of routes, such as subcutaneously, topically, orally, intraperitoneally, intradermally, intravenously, intranasally, rectally, and intramuscularly. However, it has become increasingly common to treat a variety of medical conditions by introducing a therapeutic composition directly into the tissue with the pathological conditions, such as through a catheter, to maximize the efficacy and minimize the side effects of the therapeutic composition. Such localized drug delivery is particularly needed for the treatment of brain-related diseases, cancer, and gene therapy. For example, chemotherapeutic agents are known for their toxicity and therefore it is highly desirable to have them delivered only to cancer cells.

Methods for delivering drugs to body lumens or particular target tissues may involve, for example, the use of catheters having a balloon disposed on the distal end of the catheter, with the drugs coated on the balloon surface. For example, U.S. Pat. Nos. 5,102,402 and 6,146,358 teach a balloon catheter, in which the exterior surface of the balloon is coated with drugs. The drug is delivered to the target lumen or tissue by inserting the catheter into and maneuvering it through the cardiovascular system to reach the target site. Once in the proper position, the balloon is inflated for contacting the afflicted tissue so that the drug is released and retained in the lumen or tissue. In another example, U.S. Pat. Nos. 6,409,716 and 6,364,856 teach balloon catheters with drug-embedded polymer layers coated upon the balloon surface. These medical devices allow for a rapid release of the drug from the coated polymer layer during compression of the polymer coating against the wall of the lumen as the balloon is expanded. Drug-coated medical devices of the foregoing types do, however, have certain inherent disadvantages. For example, the coating may not adhere properly to the balloon surface, thereby causing difficulties when using the device. These devices may also not reach the target sites.

Among various types of localized delivery methods, intra-arterial drug delivery is preferred for the treatment of certain types of medical conditions, particularly, brain-related disorders or cardiovascular disorders. The successful use of intra-arterial drug delivery in those cases may save a patient from potentially life-threatening surgical procedures, such as open-heart surgery. Nonetheless, the fundamental problem with intra-arterial drug delivery is that the arterial blood flow washes out the drug rapidly, thereby, decreasing the uptake of the drug by the target tissue. A number of factors may affect the efficacy of the intra-arterial drug delivery, such as the rate of the drug uptake (which is a function of drug concentration, rate of transfer across the tissue-arterial barrier, baseline blood flow, to name a few), transit time (i.e., the time of contact between arterial blood and tissue), and the elimination kinetics from the tissue. Controlling the arterial blood flow may be critical increase drug delivery efficiency. Experiments suggest that decreasing blood flow can increase the effects of some drug by 3-4 folds. However, arresting blood flow to a tissue can potentially cause ischemic injury. In addition, sustained occlusion may cause reactive increase in blood flow. Such an increase in blood flow would enhance drug elimination from the tissue once the occlusion is released.

### SUMMARY OF THE INVENTION

The present invention provides a steerable device, such as a catheter, comprising a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

In one aspect, the present invention also provides a drug delivery system which comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated by the balloon drive. In one embodiment, the drug delivery system further comprises a computerized device, wherein the computerized device controls the balloon drive.

In another aspect, the present invention provides a method for the localized delivery of an agent to a target location within a subject, comprising the steps of: (1) partially or completely arresting blood flow to the target location for a short period of time; (2) delivering the agent in bolus to the target location; and (3) partially or completely restoring blood flow to the target tissue, wherein the blood flow is arrested by occluding the artery to the target tissue. In one embodiment, the steps of (1)-(3) are repeated at least once. Preferably, the inflation and deflation of the balloon is controlled by a balloon drive. In a more preferred embodiment, the balloon drive is controlled by a computerized device. In another embodiment of the present invention, the agent, e.g., an agent for treating brain-related disorders, a chemotherapeutic agent, and a gene-therapy agent, is delivered using a catheter.

The present invention further provides a method for the localized delivery of an agent to a target location within a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated; (2) incorporating the agent into the drug delivery system; and (3) delivering the agent to the target location. In a preferred embodiment, the agent is delivered to the target location in bolus.

Additionally, the present invention provides a method for the treatment of a pathological disorder in a subject, comprising the steps of: (1) partially or completely arresting blood flow to a target tissue for a short period of time; (2) delivering a therapeutic agent in bolus; and (3) partially or completely restoring blood flow to the target tissue, wherein the target tissue has a pathological condition and the blood flow is arrested by occluding the artery to the target tissue, and wherein the therapeutic agent is delivered into the target tissue or a location within the artery which is close to the target tissue. In one embodiment, the steps of (1)-(3) are repeated at least once. Preferably, the inflation and deflation of the balloon is controlled by a balloon drive. In a more preferred embodiment, the balloon drive is controlled by a computerized device. In another embodiment of the present invention, the therapeutic agent, e.g., an agent for treating brain-related disorders, a chemotherapeutic agent, and a gene-therapy agent, is delivered using a catheter.

In another aspect, the present invention provides a method for the treatment of a pathological disorder in a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated; (2) incorporating a therapeutic agent into the drug delivery system; and (3) delivering the therapeutic agent to a target location, wherein the target location is in or close to a target tissue in the subject, wherein the target tissue has a pathological condition. In a preferred embodiment, the therapeutic agent is delivered to the target location in bolus.

Additional aspects of the present invention will be apparent in view of the description that follows.

### BRIEF DESCRIPTION OF THE FIGURES

FIG. 1 sets forth the effect of bolus configuration (concentration and volume) on the dose requirements of intracarotid propofol to produce 5 minutes of electrocerebral silence.

FIG. 2 shows the effects of bolus dose on the total dose required to produce 5 minutes of electrocerebral silence. Data from 9 animals who received 4 different doses (0.33%*0.1 ml, 0.33%*0.3 ml, 1%*0.1 ml, and 1%*0.3 ml) of propofol to produce 5 minutes of EEG silence. The total dose requirement was a function of the amount of drug in each bolus.

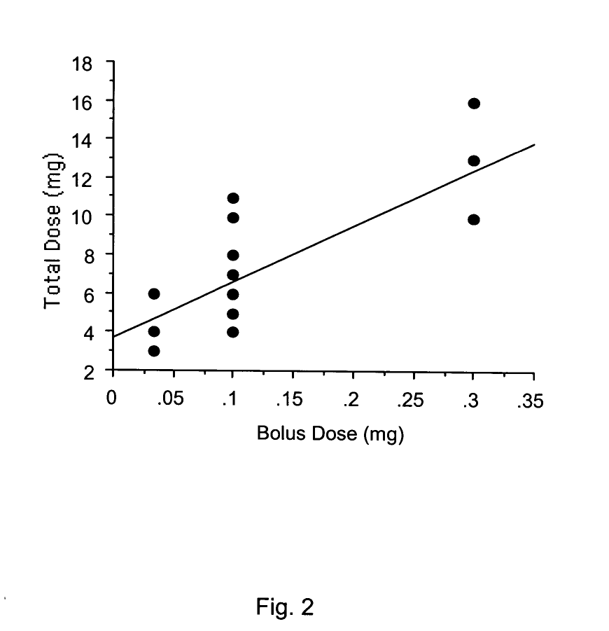

FIG. 3 depicts the effects of ventilation on cerebral blood flow (CBF) and dose-requirements. Animals were subjected to normal ventilation, hyperventilation, and hypoventilation (3 challenges/animal) to alter CBF. Data from 9 animals (27 data pionts) showing a linear correlation between cerebral blood flow changes from the baseline, the change in ventilation, and the dose required to produce 10 minutes of EEG silence with intracarotid propofol.

FIG. 4 sets forth the effect of pentothal 3 mg on the duration of EEG silence while administering the drug during flow arrest (i.e., a severe reduction in CBF with systemic hypotension achieved with esmolol and adenosine). Pentothal 1 and 3 were injected under normotensive conditions while the second injection (pentothal-2) was made during flow arrest, N=9.

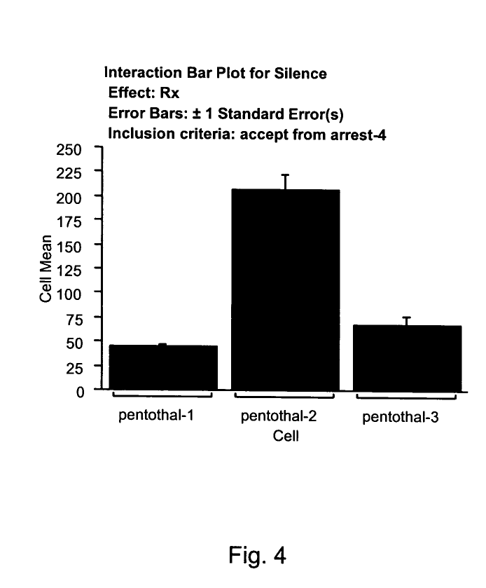

FIG. 5 shows the increased blood flow by intra-arterial verapamil, a cerebral vasodilator, increases the dose requirements of intracarotid propofol to produce 10 minutes of EEG silence. FIG. 5a: The doses of intracarotid propofol required were significantly increased with co-administration of intracarotid verapamil (n=14, P=0.04). The data include three animals with hypotension and a decrease in cerebral blood flow. FIG. 5b: The dose requirements for intracarotid propofol were significantly increased with the increase in blood flow with IA verapamil. All of the experimental animals showed a decrease in blood flow (<100%) and low IC propofol requirements. There was a strong correlation between blood flow and the dose of the drug (r=0.75, P=0.0021).

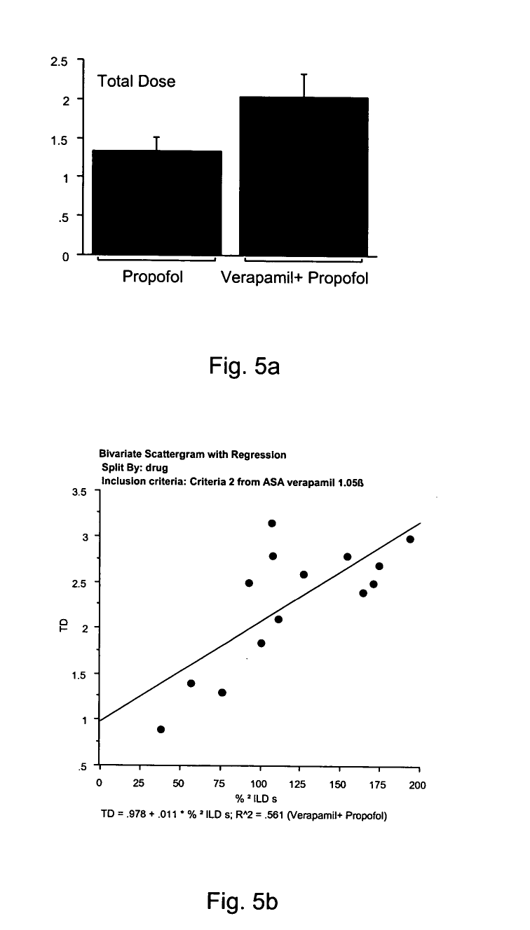

FIG. 6 depicts the effect of different concentration of propofol (0.33% and 1%) on the duration of electrocerebral silence before (0.33%-1 and 1%-1), during (Arrest-0.33% and Arrest-1%) and after (0.33%-2 and 1%-2). Severe hypotesion lasted 3-5 minutes and flow arrest (to ˜25% of baseline) lasted for 20 seconds. Changing concentration increased the duration of EEG silence by 2-4 folds (P=0.02, NS on post-hoc testing) while flow arrest increased the duration of EEG silence 15-20 folds (n=8, P<0.0001).

FIG. 7 illustrates the arterial occlusion drug delivery catheter and a drug delivery system comprising the same.

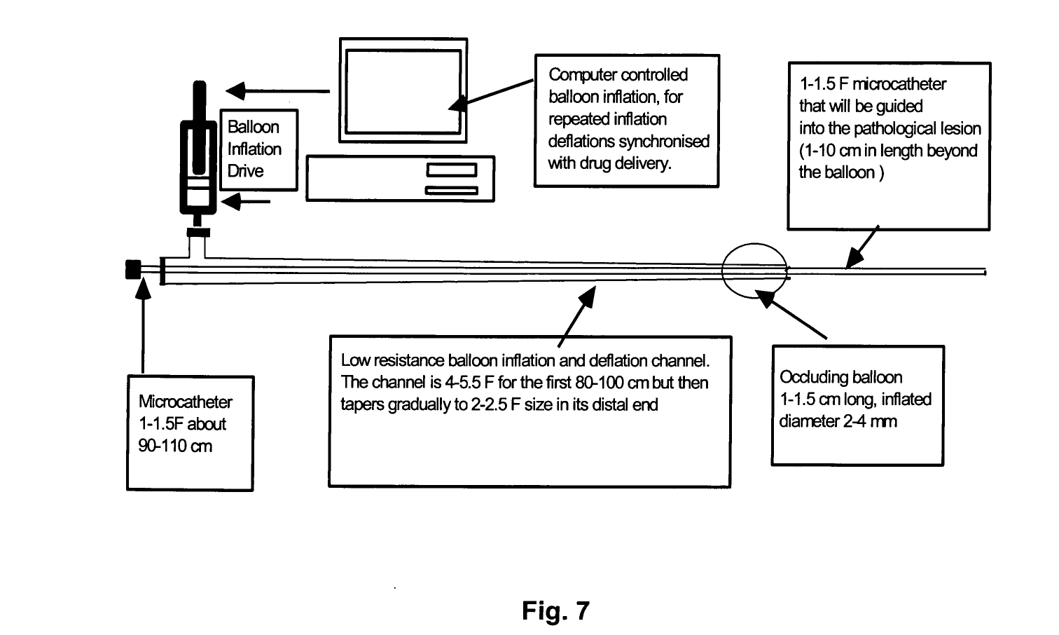

### DETAILED DESCRIPTION OF THE INVENTION

As used herein and in the appended claims, the singular forms “a,” “an” and “the” include plural references unless the content clearly dictates otherwise. Thus, for example, reference to “an agent” includes a plurality of such agents and reference to “the vector” is a reference to one or more vectors and equivalents thereof known to those skilled in the art, and so forth. All publications, patent applications, patents and other references mentioned herein are incorporated by reference in their entirety.

The present invention provides a steerable device, e.g., a catheter, comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated. In one embodiment, the proximal double-lumen assembly is about 4-5.5 French in diameter for the first 80-100 cm. In the distal end, preferably, the distal 8-15 cm, the diameter of the proximal double-lumen assembly is gradually narrowed to about 2-3 French. The micro-catheter is extended beyond the distal end of the balloon for a variable length, preferably, 1-10 cm beyond the distal end of the balloon. In one embodiment, the micro-catheter is about 1-2 French in diameter, preferably, about 1.2-1.5 French in diameter. In another embodiment, the balloon is about 1-1.5 cm in length. The balloon of the present invention can be rapidly inflated or deflated, optimally, in about 1 second.

The steerable device, e.g., a catheter, is preferably made of materials which render the steerable device strong enough to withstand repeated inflation and deflation of the balloon, flexible enough to negotiate the curve of blood vessels and having low frictional resistance and thrombogenic potential. The material may be any suitable material with high tensile strength, such as, Teflon, nylon, polyurethane, and polyethylene. In one embodiment, to increase maneuverability and decrease the risk of thromboembolism, the steerable device has a surface coating. In a preferred embodiment, the surface coating is a hydrophilic surface coating.

In one aspect, the present invention provides a drug delivery system which comprises a steerable device and a balloon drive, wherein the steerable device comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated. In one embodiment, the proximal double-lumen assembly is about 4-5 French in diameter for the first 80 cm. In the distal end, preferably, the distal 10 cm, the diameter of the proximal double-lumen assembly is gradually narrowed to about 2-3 French. The micro-catheter is extended beyond the distal end of the balloon for a variable length, preferably, 1-10 cm beyond the distal end of the balloon. In a preferred embodiment, the micro-catheter is about 1-2 French in diameter, more preferably, about 1.2-1.5 French in diameter. In another embodiment, the balloon is about 1-1.5 cm in length. The balloon of the present invention can be rapidly inflated or deflated, optimally, in about 1 second.

The steerable device, e.g., a catheter, is preferably made of materials which render the steerable device strong enough to withstand repeated inflation and deflation of the balloon, flexible enough to negotiate the curve of blood vessels, and having low frictional resistance and thrombogenic potential. The material may be any suitable material with high tensile strength, such as, Teflon, nylon, polyurethane and polyethylene. In one embodiment, to increase maneuverability and decrease the risk of thromboembolism, the steerable device has a surface coating, preferably, a hydrophilic coating.

The inflation and deflation of the balloon is controlled by the balloon drive. The balloon drive may be any device which is capable of rapidly inflating or deflating the balloon. In one embodiment, the balloon drive inflates or deflates the balloon in less than 20 seconds, preferably, in less than 5 seconds, and more preferably, in about 1 second. The balloon drive may use any suitable liquid or gas to inflate the balloon. In a preferred embodiment, the balloon is inflated by a radio-opaque low-viscosity fluid. The fluid based balloon distention mechanism decreases the time required to inflate a balloon.

The drug delivery system may further comprise a computerized device to control the balloon drive. The computerized device may be any computing system suitable for controlling the balloon drive. The computerize device may be a stand-alone computer, which is functionally connected to the balloon drive, or integrated with the balloon drive. In either case, the computer is capable of receiving external and/or internal input and transferring the input into signals to control the behavior of the balloon drive. The input information may be any information that may contribute to the manipulation of the function of the balloon drive. The primary inputs are parameters used by the computerized device to control the balloon drive, such as the frequency, duration and volume of inflation/deflation.

The present invention further provides a method for the localized delivery of an agent to a target location within a subject, comprising the steps of: (1) partially or completely arresting blood flow to the target location for a short period of time; (2) delivering the agent in bolus to the target location; and (3) partially or completely restoring blood flow to the target tissue, wherein the blood flow is arrested by occluding the artery to the target tissue.

As used herein, the “subject” is an animal, preferably a mammal including, without limitation, a cow, dog, human, monkey, mouse, pig or rat. The term “agent,” as used herein, shall include any protein, polypeptide, peptide, nucleic acid (including DNA, RNA, and genes), antibody and fragment thereof, molecule, compound, antibiotic, drug and any combinations thereof. The agent of the present invention may have any activity, function or purpose. By way of example, the agent may be a diagnostic agent, a labeling agent, a preventive agent, or a therapeutic or pharmacologic agent.

As used herein, a “diagnostic agent” is an agent that is used to detect a disease, disorder or illness or is used to determine the cause thereof. As further used herein, a “labeling agent” is an agent that is linked to, or incorporated into, a cell or molecule, to facilitate or enable the detection or observation of that cell or molecule. By way of example, the labeling agent of the present invention may be an imaging agent or detectable marker and may include any of those radioactive labels known in the art. For instance, the labeling agent may be a radioactive marker, including a radioisotope, such as a low-radiation isotope. The radioisotope may be any isotope that emits detectable radiation, and may include ³⁵S, ³²P, ³H, radioiodide (¹²⁵I or ¹³¹I) or ⁹⁹ mTc-pertechnetate (⁹⁹mTcO4⁻). Radioactivity emitted by a radioisotope can be detected by techniques well known in the art.

Additionally, as used herein, the term “preventive agent” refers to an agent, such as a prophylactic, that helps to prevent a disease, disorder or illness in a subject. As further used herein, the term “therapeutic” refers to an agent that is useful in treating a disease, disorder or illness (e.g., a neoplasm) in a subject. In one embodiment, the anti-neoplasm agent used in a method to prevent and treat a neoplasm is an antibody. In a preferred embodiment, the antibody is preferably a mammalian antibody (e.g., a human antibody) or a chimeric antibody (e.g., a humanized antibody). More preferably, the antibody is a human or humanized antibody. As used herein, the term “humanized antibody” refers to a genetically-engineered antibody in which the minimum portion of an animal antibody (e.g., an antibody of a mouse, rat, pig, goat or chicken) that is generally essential for its specific functions is “fused” onto a human antibody. In general, a humanized antibody is 1-25%, preferably 5-10%, animal; the remainder is human. Humanized antibodies usually initiate minimal or no response in the human immune system. Methods for expressing fully human or humanized antibodies in organisms other than human are well known in the art (see, e.g., U.S. Pat. No. 6,150,584, Human antibodies derived from immunized xenomice; U.S. Pat. No. 6,162,963, Generation of xenogenetic antibodies; and U.S. Pat. No. 6,479,284, Humanized antibody and uses thereof). In one embodiment of the present invention, the antibody is a single-chain antibody. In a preferred embodiment, the single-chain antibody is a human or humanized single-chain antibody. In another preferred embodiment of the present invention, the antibody is a murine antibody.

In one embodiment of the present invention, the therapeutic agent, such as an anti-neoplasm agent, may be a nucleic acid (e.g., plasmid). The nucleic acid may encode or comprise at least one gene-silencing cassette, wherein the cassette is capable of silencing the expression of genes that are essential or important for the survival or proliferation of pathogens or neoplastic cell. It is well understood in the art that a gene may be silenced at a number of stages including, without limitation, pre-transcription silencing, transcription silencing, post-transcription silencing, translation silencing and post-translation silencing. The nucleic acid may also encode polypeptides or other types of biological molecules which are capable of compensating or correcting a defect in a subject.

In one embodiment of the present invention, the gene-silencing cassette encodes or comprises a post-transcription gene-silencing composition, such as antisense RNA or RNAi. Both antisense RNA and RNAi may be produced in vitro, in vivo, ex vivo, or in situ.

For example, the therapeutic agent of the present invention, e.g., an anti-neoplasm or anti-infection agent, may be an antisense RNA. Antisense RNA is an RNA molecule with a sequence complementary to a specific RNA transcript, or mRNA, whose binding prevents further processing of the transcript or translation of the mRNA. Antisense molecules may be generated synthetically or recombinantly with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein, et al., Oligodeoxynucleotides as inhibitors of gene expression: a review. Cancer Res., 48:2659-68, 1998).

Antisense molecules designed to bind to the entire mRNA may be made by inserting cDNA into an expression plasmid in the opposite or antisense orientation. Antisense molecules may also function by preventing translation initiation factors from binding near the 5′ cap site of the mRNA, or by interfering with interaction of the mRNA and ribosomes (e.g., U.S. Pat. No. 6,448,080, Antisense modulation of WRN expression; U.S. Patent Application No. 2003/0018993, Methods of gene silencing using inverted repeat sequences; U.S. Patent Application No. 2003/0017549, Methods and compositions for expressing polynucleotides specifically in smooth muscle cells in vivo; Tavian, et al., Stable expression of antisense urokinase mRNA inhibits the proliferation and invasion of human hepatocellular carcinoma cells. Cancer Gene Ther., 10:112-20, 2003; Maxwell and Rivera, Proline oxidase induces apoptosis in tumor cells and its expression is absent or reduced in renal carcinoma. J. Biol. Chem., e-publication ahead of print, 2003; Ghosh, et al., Role of superoxide dismutase in survival of Leishmania within the macrophage. Biochem. J, 369:447-52, 2003; and Zhang, et al., An anti-sense construct of full-length ATM cDNA imposes a radiosensitive phenotype on normal cells. Oncogene, 17:811-8, 1998).

In one embodiment, oligonucleotides antisense to a biological molecule, such as a member of the infection/neoplasm-related signal-transduction pathways/systems, may be designed based on the nucleotide sequence of the member of interest. For example, a partial sequence of the nucleotide sequence of interest (generally, 15-20 base pairs), or a variation sequence thereof, may be selected for the design of an antisense oligonucleotide. This portion of the nucleotide sequence may be within the 5′ domain. A nucleotide sequence complementary to the selected partial sequence of the gene of interest, or the selected variation sequence, then may be chemically synthesized using one of a variety of techniques known to those skilled in the art including, without limitation, automated synthesis of oligonucleotides having sequences which correspond to a partial sequence of the nucleotide sequence of interest, or a variation sequence thereof, using commercially-available oligonucleotide synthesizers, such as the Applied Biosystems Model 392 DNA/RNA synthesizer.

Once the desired antisense oligonucleotide has been prepared, its ability to prevent or treat diseases, such as neoplasm, then may be assayed. For example, the antisense oligonucleotide may be administered to a subject, such as a mouse or a human, and its effects on the disease may be determined using standard clinical and/or molecular biology techniques, such as Western-blot analysis and immunostaining.

It is within the confines of the present invention that antisense oligonucleotides may be linked to another agent, such as an anti-infection, an anti-neoplastic drug, or an agent which facilitate the transportation of the antisense oligonucleotides into a cell (e.g., penetratin, transportan, pIsl, TAT, pVEC, MTS, and MAP). Moreover, antisense oligonucleotides may be prepared using modified bases (e.g., a phosphorothioate), as discussed above, to make the oligonucleotides more stable and better able to withstand degradation.

The therapeutic agent of the present invention also may be an interfering RNA, or RNAi, including small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi is produced in vivo by an expression vector containing a gene-silencing cassette coding for RNAi (see, e.g., U.S. Pat. No. 6,278,039, C. elegans deletion mutants; U.S. Patent Application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes). In another embodiment of the present invention, RNAI is produced in vitro, synthetically or recombinantly. Methods of making and transferring RNAi are well known in the art (see, e.g., Ashrafi, et al., Genome-wide RNAi analysis of Caenorhabditis elegans fat regulatory genes. Nature, 421:268-72, 2003; Cottrell, et al., Silence of the strands: RNA interference in eukaryotic pathogens. Trends Microbiol., 11:37-43, 2003; Nikolaev, et al., Parc. A Cytoplasmic Anchor for p53. Cell, 112:29-40, 2003; Wilda, et al., Killing of leukemic cells with a BCR/ABL fusion gene RNA interference (RNAi). Oncogene, 21:5716-24, 2002; Escobar, et al., RNAi-mediated oncogene silencing confers resistance to crown gall tumorigenesis. Proc. Natl. Acad. Sci. USA, 98:13437-42, 2001; and Billy, et al., Specific interference with gene expression induced by long, double-stranded RNA in mouse embryonal teratocarcinoma cell lines. Proc. Natl. Acad. Sci. USA, 98:14428-33, 2001).

Once the desired RNAi has been prepared, its ability to prevent or treat diseases, such as neoplasm, then may be assayed. For example, the RNAi may be administered to a subject, such as a mouse or a human, and its effects on the disease may be determined using standard clinical and/or molecular biology techniques, such as Western-blot analysis and immunostaining.

It is within the confines of the present invention that an RNAi may be linked to another agent, such as an anti-infection, an anti-neoplastic drug, or an agent which facilitate the transportation of the antisense oligonucleotides into a cell (e.g., penetratin, transportan, pIsl, TAT, pVEC, MTS, and MAP). Moreover, an RNAi may be prepared using modified bases (e.g., a phosphorothioate), as discussed above, to make it more stable and better able to withstand degradation.

The agent may also be a pharmaceutical composition comprising the a therapeutic agent and a pharmaceutically acceptable carrier. The pharmaceutically acceptable carrier must be “acceptable” in the sense of being compatible with the other ingredients of the composition, and not deleterious to the recipient thereof. The pharmaceutically acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, viscosity-increasing agents, etc. If necessary, pharmaceutical additives, such as antioxidants, may also be added. Examples of acceptable pharmaceutical carriers include glycerin, lactose, magnesium stearate, saline, sodium alginate, sucrose, and water, among others.

The composition of the present invention may be prepared by methods well known in the pharmaceutical arts. For example, the composition may be brought into association with a carrier or diluent, as a suspension or solution. Optionally, one or more accessory ingredients (e.g., buffers, surface active agents, and the like) also may be added.

The pharmaceutical composition is provided in an amount effective to treat the disorder in a subject to whom the composition is administered. As used herein, the phrase “effective to treat the disorder” means effective to ameliorate or minimize the clinical impairment or symptoms resulting from the infectious disease or neoplasia. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia; or by limiting, suspending, terminating, or otherwise controlling the proliferation of cells in the neoplasm.

The amount of pharmaceutical composition that is effective to treat infectious diseases and neoplasia in a subject will vary depending on the particular factors of each case, including, for example, the type or stage of the infection or neoplasia, and the severity of the subject's condition. These amounts can be readily determined by a skilled artisan.

In accordance with the method of the present invention, the pharmaceutical composition may be administered to a subject, either alone or in combination with one or more other therapeutic agents, such as antibiotics or antineoplastic drugs. Examples of antibiotics with which the pharmaceutical composition may be combined include, without limitation, penicillin, tetracycline, bacitracin, erythromycin, cephalosporin, streptomycin, vancomycin, D-cycloserine, fosfomycin, cefazolin, cephaloglycin, cephalexin, amphotericin B, gentamicin, tobramycin, kanamycin, and variants and derivatives thereof. Examples of antineoplastic drugs with which the pharmaceutical composition may be combined include, without limitation, carboplatin, cyclophosphamide, doxorubicin, etoposide and vincristine. The pharmaceutical composition of the present invention may also be administered to a subject together with an agent which is capable of improving the uptake of the pharmaceutical composition by the target tissue. For example, serotonin may be used to enhance arterial permeability and thus facilitate the transition of the therapeutic composition from artery to the target tissue.

Under certain circumstances, it is necessary to repeat the steps of (1)-(3) of the method of the present invention at least once. For example, the target tissue may be very sensitive to ischemic injury and thus shall not be subject to long-term blood occlusion. It is therefore preferable to repeat steps (1)-(3) such that, on the one hand, enough agents such as therapeutic drugs can be delivered to the target tissue; on the other hand, damages caused by ischemia-reperfusion may be minimized.

In one embodiment, the blood flow is arrested by inflating a balloon and restored by deflating the balloon. In another embodiment, the blood flow is arrested through a balloon together with a blood flow arresting pharmaceutical composition, such as adenosine and esmolol. Complete blood flow arrest is not always necessary for efficient drug delivery. A transient (e.g., 20-30 seconds) flow decrease to about 25% of baseline value is sufficient to enhance significantly the delivery of drug. In a preferable embodiment, the balloon is rapidly inflated and/or deflated, such as within about 1 second. In another embodiment, the duration of the balloon inflation is about 10-150 seconds. The inflation time depends on the ability of the tissue to with stand reduced blood flows. For organs like the brain the inflation time will be short (10-150 seconds) but for liver and heart it could be much longer (several minutes). It is desirable to employ a computer-controlled balloon drive to regulate the inflation and deflation of the balloon.

The agent may be delivered using a catheter. In one embodiment, the catheter comprises a proximal double-lumen assembly and a balloon, wherein the first lumen of the double-lumen assembly is a micro-catheter and the second lumen of the double-lumen assembly is a larger lumen for inflating and deflating the balloon and wherein the balloon can be rapidly inflated and deflated.

The agent delivered may be any therapeutic or diagnostic agent for the treatment or diagnosis of pathological conditions including, without limitation, agents for treating brain-related disorders, chemotherapeutic agents, and gene-therapy agents.

In one embodiment, the target location is in or close to a tissue in the subject, wherein the tissue has a pathological condition. Preferably, the target location is the artery in or near brain, a tumor, or a tissue in need of gene-therapy, such as carotid. In a preferred embodiment, the subject is a mammal, including human.

The devices and methods of the present invention are particular suitable for delivering drugs to the brain. The arteries in the brain are end-arteries, i.e., they do not join each other after they branch off from the parent arteries. Thus proximal arterial occlusion can effectively decrease blood flow in the distal regions of the brain. Furthermore, the devices and methods of the present invention may significantly improve cancer chemotherapy. Chemotherapeutic agents are generally poorly absorbed when given intra-arterially. The controlled-arterial occlusion drug delivery technique provided by the present invention will be very useful for efficient delivery of chemotherapeutic agents and thus decreasing the dose of chemotherapeutic agents needed and the systemic complications caused by these agents, which are generally highly toxic. Additionally, intra-arterial occlusion drug therapy could play a critical role in delivering gene therapy agents, such as viral vectors, liposomes and gene fragments.

The present invention also provides a method for the localized delivery of an agent to a target location within a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon and wherein the balloon can be rapidly inflated and deflated; (2) incorporating the agent into the drug delivery system; and (3) delivering the agent to the target location.

The inflation and deflation of the balloon is controlled by the balloon drive, which, preferably, is controlled by a computerized device. The computerized device may be any computing system suitable for controlling a balloon drive. In one embodiment, the computerized device is a stand-alone computer system, with an input device, user-machine interface, and is functionally connected to the balloon drive. In another embodiment, the computerized device is a sub-component of a component of the drug delivery system, wherein the component further comprises the balloon drive. Depending on specific situations, such as the condition of the subject, the type of the target tissue, the purpose of the operation, the characteristic of the agent, different parameters should be used to control the behavior of the balloon drive and consequently, the inflation and deflation of the balloon. In one embodiment, the primary parameters used by the computerized device to control the balloon drive are the frequency, duration, and volume of inflation/deflation. The parameters may be manually inputted through a user-computer interface and an inputting device, or imported from a database, such as a medical expert system. The frequency of balloon inflation and deflation will be a function of a number of factors including, without limitation, the rate of efflux of the drug from the tissue, the duration of inflation, the type of the tissue, the type of the agent and the characteristics of reactive hyperemia in the tissue. The duration of the inflation will be a function of the risk of ischemic injury to the tissue, typically, between about 2-600 seconds, preferably, between about 5-100 seconds, more preferably, between about 15-60 seconds.

The balloon may be inflated by any suitable gas or liquid. In one embodiment, the balloon is inflated by fluid. The use of fluid will decrease the time required to inflate the balloon. Preferably, a radio-opaque low viscosity fluid is used to inflate the balloon because it will facilitate the imaging and monitoring of the performance of the balloon and the catheter.

In another embodiment, the agent used in the present method is a therapeutic or diagnostic agent, such as an agent for treating brain-related disorders, a chemotherapeutic agent, and a gene-therapy agent. To facilitate the effective delivery of the agent, it is desirable to have the balloon inflated to arrest partially or completely the blood flow to the target location. The target location is in or close to a tissue in the subject, wherein the tissue has a pathological condition, for example, the target location may be the artery (e.g., carotid) in or near brain, a tumor or a tissue in need of gene-therapy.

In a preferred embodiment, the agent is delivered to the target location in bolus. Computer simulations indicate that the efficacy of intra-arterial drug delivery is inversely affected by regional blood flow. For example, high blood flow creates a stable fluid flow system. A stable fluidic flow pattern can trap drugs within a sub-stream, resulting in streaming of drugs. Streaming generates heterogeneous distributions of drugs within the target tissue. There are variations in tissue drug concentrations as well as distribution after continuous infusions. Such unpredictability is therapeutically undesirable. Therefore, bolus delivery of drugs is more likely to generate predictable drug concentrations in the target tissue than infusions.

The present invention further provides a method for the localized delivery of an agent to a target location within a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon and wherein the balloon can be rapidly inflated and deflated; (2) incorporating the agent into the drug delivery system; (3) occluding blood flow to the target location by inflating the balloon; (4) delivering the agent in bolus to the target location; and (5) deflating the balloon after a short period of time.

In one aspect, the present invention provides a method for the treatment of a pathological disorder in a subject, comprising the steps of: (1) partially or completely arresting blood flow to a target tissue for a short period of time; (2) delivering a therapeutic agent in bolus; and (3) partially or completely restoring blood flow to the target tissue, wherein the target tissue has a pathological condition and the blood flow is arrested by occluding the artery to the target tissue, and wherein the therapeutic agent is delivered into the target tissue or a location within the artery which is close to the target tissue. In one embodiment, the steps of (1)-(3) are repeated at least once to ensure sufficient drug delivery and minimize the ischemic injury. In another embodiment, the target tissue is brain, a tumor, or a tissue in need of gene-therapy.

The blood flow may be arrested by inflating a balloon and restored by deflating the balloon. In one embodiment, the balloon is inflated or deflated in about 1 second using a balloon drive, which is subsequently controlled by a computerized device.

The therapeutic agent may be delivered using a catheter. In one embodiment, the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

In another aspect, the present invention further provides a method for the treatment of a pathological disorder in a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated; (2) incorporating a therapeutic agent into the drug delivery system; and (3) delivering the therapeutic agent to a target location, wherein the target location is in or close to a target tissue in the subject, wherein the target tissue has a pathological condition. In one embodiment, the catheter is made of a material with high tensile strength, such as Teflon, nylon, polyurethane, and polyethylene. The catheter may have a surface coating, preferably a hydrophilic surface coating. In another embodiment, the balloon inflation partially or completely blocks the blood flow to the target location. A balloon drive may be employed to control the inflation and deflation of the balloon. The balloon drive may subsequently be put under control of a computerized device.

The therapeutic agent may be any therapeutic agent suitable for the treatment of the pathological condition in the target tissue, such as an agent for treating brain-related disorders, a chemotherapeutic agent, and a gene-therapy agent. Preferably, the therapeutic agent is delivered to a target location, which is the artery in or near brain, a tumor, or a tissue in need of gene-therapy. In one embodiment, the therapeutic agent is delivered to the target location in bolus.

The present invention also teaches a method for the treatment of a pathological disorder in a subject, comprising the steps of: (1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated; (2) incorporating a therapeutic agent into the drug delivery system; (3) occluding blood flow to the target location by inflating the balloon; (4) delivering the therapeutic agent to the target location; and (5) deflating the balloon after a short period of time.

### EXAMPLES

The following examples illustrate the present invention, which are set forth to aid in the understanding of the invention, and should not be construed to limit in any way the scope of the invention as defined in the claims which follow thereafter.

### Example 1

### Materials and Methods

After the approval of the protocol by the institution's animal care and use committee, the study was conducted on New Zealand White rabbits (1.5-2.0 kg. in weight). The animals were given full access to food and water prior to the experiment. The animals were sedated with an intramuscular ketamine (50 mg/kg). Intravenous access was obtained through an earlobe vein. Hydrocortisone 10 mg was given after the placement of an intravenous line, as it prevents hypotension, which sometimes occurs after surgical intervention in this animal species. Subsequently, the animal received 0.2 ml boluses of intravenous propofol (Diprivan® 1%, Astra Zeneca Pharmaceutical LP, Wilmington, Del.) as needed for maintaining adequate depth of anesthesia prior to tracheostomy. After infiltration of the incision site with local anesthetic, 0.25% bupivacaine with 1:200,000 epinephrine, a tracheotomy was undertaken for placement of endotracheal tube for mechanical ventilation by a Harvard small animal ventilator (Harvard Apparatus Inc., South Natick, Mass.). End-tidal CO₂ (ETCO₂) was continuously monitored with Novametrix Capnomac monitor (Novametrix Medical Systems Inc., Wallingford, Conn.). After securing the airway, anesthesia was maintained with intravenous infusion of propofol 1-2 ml/kg/hr, fentanyl 1-2 μg/kg/hr and vecuronium bromide 10-20 μg/kg/hr. A femoral arterial line was placed for monitoring mean arterial blood pressure (MAP).

The right common carotid artery was dissected in the neck and cannulated using a 20 cm-long PE-50 tubing (Becton Dickinson and Co., Spark, Md.). Correct identification of the internal carotid artery and its isolation was confirmed by the retinal discoloration test (Joshi et al., Retinal Discoloration Test. J Cerebral Blood Flow Metabolism 24:305-3082004, 2004). Briefly, this test entails injection of 0.1-0.2 ml of 0.05% indigocarmine-blue, which changes the retinal reflex from red to blue when the internal carotid artery is correctly identified.

An esophageal temperature probe was used to monitor core temperature (e.g., Nova Therm, Novamed Inc., Rye, N.Y., or Mon-a-therm, 400H, Mallinckrodt Anesthesia Products, St. Louis, Mo.). The animal's temperature was kept constant between 37±1.0° C. using an electrically heated blanket. An intravenous infusion of fluid was given at 10 ml/kg/hr through an IVAC pump (IVAC 599 volumetric pump, IVAC Co., San Diego, Calif.). The intravenous infusion consisted of three fluids: ringer lactate, 5% dextrose, and 5% albumin mixed in a ratio of 3:1:1, respectively. Electroencephalographic recording (EEG), MAP, ETCO₂ and laser Doppler flows were continuously recorded on a computer using Powerlab software (AD Instruments Inc., Grand Junction, Colo.).

To measure cerebral blood flow (CBF), two laser Doppler probes (Probe# 407-1, Perimed Inc. Jarfalla, Sweden) were placed on either hemisphere. For probe placement, the animals were turned prone and positioned on a stereotactic frame. The skull was exposed through a midline incision. A 5×4 mm area of the skull was shaved with a drill, slightly anterior to the bregma and 1 mm lateral to the mid-line. The skull was shaved to expose the inner table, such that the cortical vessels could be seen through a fine layer of bone as described in literature (Morita-Tsuzuki, et al., Vasomotion in the rat cerebral microcirculation recorded by laser-Doppler flowmetry. Acta Physiologica Scandinavica 146:431-9, 1992). The probes were maneuvered to obtain a laser Doppler blood flow reading of 50-250 perfusion units (PU). Once the optimum site of placement was identified, the probes were secured within plastic retainers, and glued to the skull. Satisfactory probe placement was judged by an abrupt increase in probe reading during intracarotid injection of a small volume of saline (0.1 ml). Laser Doppler blood flow measurement technique provided a relative measure of blood flow changes in the tissue, therefore, laser Doppler blood flow values were normalized to the baseline value and were expressed as %-change from baseline value.

Fronto-parietal leads were placed and used to monitor the bilateral electrocerebral activity. Electrocerebral activity was monitored using standard stainless steel needle electrodes (impedance is <10 k Ohms). The frontal and the parietal needle electrodes were secured to the skull by small stainless steel screws. The neutral electrode placed in the temporalis muscle. Fronto-parietal electro-encephalographic signals were recorded using bioamplifier (ML136, AD Instruments, Grand Junction, Colo.), with a range of 100 mV, and electrocerebral activity (or electroencephalogram) recording mode having a pass-band 0.3 to 60 Hz. Analog data was sampled at 100 Hz per channel with an analog to digital converter, and displayed using the Chart 4.2 program (AD Instruments, Grand Junction, Colo.).

Electrocerebral silence was defined operationally, using a reference recording obtained with an identical recording technique from a known brain dead preparation following administration in intravenous KCl (Illievich, et al., Effects of hypothermia or anesthetics on hippocampal glutamate and glycine concentrations after repeated transient global cerebral ischemia. Anesthesiol. 80:177-86, 1994). A burst suppression pattern was evident during recovery from electrocerebral silence that was characterized by transient bursts of electrocerebral (or EEG) activity in the 30-50 μV range spaced with intervening period of electrocerebral silence. Electrocerebral recovery was defined as the return of electrocerebral activity with amplitudes and frequency composition comparable to baseline as judged by visual inspection (La Marca, et al., Cognitive and EEG recovery following bolus intravenous administration of anesthetic agents. Psychopharmacol. (Berl) 120:426-32, 1995). Total recovery time was defined as time between onset of electrocerebral silence after last injection and upon electrocerebral recovery. Post-drug silence time was the duration of time between the injection of last bolus to the return of detected EEG activity, generally a burst suppression pattern. Post-silence recovery time was described as the time between the onset of burst suppression to the return of EEG morphology comparable to the normal. Hemodynamic and cerebral blood flow parameters for each drug challenge were evaluated at three stages of the experiment: (i) at baseline; (ii) EEG silence with propofol boluses; (iii) electrocerebral recovery.

### Example 2

### Preliminary and Definitive Studies and Data Analysis for the Examination of Intracarotid Bolus Drug Delivery

In the preliminary studies the inventor determined the dose requirement of IC propofol in eight rabbits ranging from a concentration of 0.25, 0.5, and 1% and volumes of 0.05, 0.1, 0.2, and 0.4 ml. Ten doses were tested in each animal. These doses were aimed to produce Ten minutes of EEG silence in these animals. To determine the loading dose, bolus doses were delivered every 10 second till 10 second of electrocerebral silence was obvious. Thereafter, maintenance doses were delivered when electrocerebral activity was recovered to greater than 5% of baseline amplitude. The total dose was a sum of loading and maintenance doses. The repeat challenges were undertaken after recovery of EEG amplitude and mean arterial pressure. During the preliminary studies the inventor observed that volume of 0.4 ml with 0.5% propofol produced EEG silence in all cases. The preliminary studies also suggested that multiple experiments were possible in the same animal due to a relatively rapid recovery of EEG activity after IC propofol. The recovery of EEG activity, as well as the systemic changes (i.e., blood pressure) was complete within 10 minutes of last intracarotid injection.

Based on the preliminary experiments, the inventor designed the study protocol so as to undertake repeat experiments on the same animal. Three doses (0.33 mg, 1.0 mg, and 3 mg) were generated by varying the concentrations (0.33% and 1%) and the volume (0.1 and 0.3 ml) of the drug. These doses were randomized so that all doses were administered first, second, third, and fourth for equal number of times. Compared to the preliminary studies, the inventor reduced the period of EEG silence from 10 to 5 minutes to decrease the amount of intracarotid propofol. There was 30 minutes period of rest between each intracarotid drug challenge. The loading, maintenance and total doses were determined, as described above.

The data is presented as mean±standard deviation. The hemodynamic and laser Doppler flow data recorded at three time points (baseline, silence and recovery). A P value of <0.0083 was considered significant between the four challenges (0.33%*0.1 ml, 0.33%*0.3 ml, 1%*0.1 ml, 1%*0.3 ml). A P<0.0167 was considered significant between the three stages of each challenge (baseline, drug, recovery) that was evaluated by ANOVA repeated measures with Bonferroni Dunn test for multiple comparisons. Linear regression analysis was used to determine the relation between bolus dose and dose requirements as well as electrocerebral parameters.

Discussed below are results obtained by the inventor in connection with the experiments of Examples 1-2:

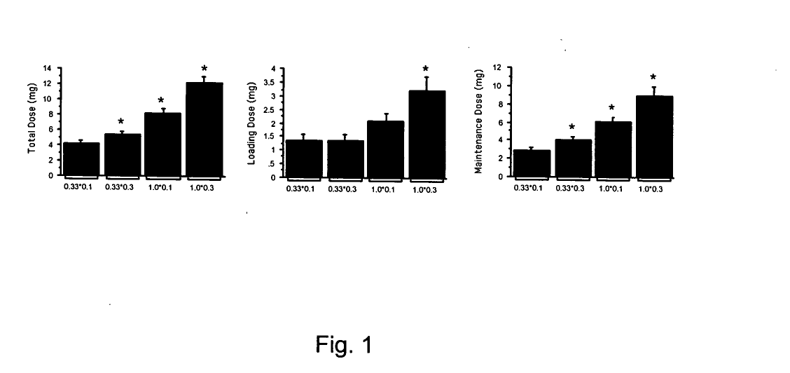

The study was conducted on eight rabbits and satisfactory data were obtained from all animals. The total dose of propofol required to produce five minutes of EEG silence was directly related to the bolus dose, x=3.6+29*y, n=32, r=0.724, P<0.0001. Both the concentration of the drug and the volume of the bolus affected the total dose requirements (FIG. 1). There was a three-fold variation in the dose requirement between the lowest and the highest bolus dose, i.e., between 0.33%*0.1 ml vs. 1%*0.3 ml from 4.3±1 to 12.3±2.1 mg, P<0.0001. The total recovery time (x=154+38*y, n=32, r²=4, P<0.0001) and the post-drug silence time (x=59+34*y, n=32, r²=0.4, P<0.0001) were a direct function of the bolus dose. However, post-silence recovery time was not significantly different with the different boluses (Table I) and had no correlation with the bolus dose x=95+4.5*y, n=32, r²=0.022, P=0.42.

TABLE I
The Effect Bolus Configuration of Propofol on the Dose
Requirements and EEG Parameters

| n = 8 | 0.33% * 0.1 ml | 0.33% * 0.3 ml | 1.0% * 0.1 ml | 1.0% * 0.3 ml |
|---|---|---|---|---|
| Total Dose (mg) | 4.3 ± 1.0 | 5.7 ± 1.2* | 8.3 ± 1.6*@ | 12.3 ± 2.1,*@# |
| Loading Dose (mg) | 1.4 ± 0.5 | 1.7 ± 0.5 | 2.1 ± 0.6* | 3.3 ± 1.4* |
| Maintenance Dose | 2.9 ± 0.7 | 4.0 ± 1.1* | 6.1 ± 1.6* | 9.0 ± 2.8*@# |
| (mg) | | | | |
| Total Recovery | 64 ± 31 | 108 ± 32 | 86 ± 33 | 159 ± 47*# |
| Time (s) | | | | |
| Post-drug Silence (s) | 156 ± 44 | 207 ± 47 | 194 ± 37 | 266 ± 61*# |
| Post-Silence | 92 ± 22 | 99 ± 33 | 108 ± 43 | 107 ± 38 |
| Recovery Time (s) | | | | |

Symbols:
*significant post-hoc differences between challenges (P < 0.0083) between groups.
Significant post hoc differences between:
*from 0.33% * 0.1 ml,
@from 0.33% * 0.3 ml,
#from 1.0% * 0.1 ml.

The baseline hemodynamic parameters were comparable across animals and across the four drug challenges (Table II and III). There was no difference in the hemodynamic effects of the four challenges (Table II and III). The MAP decreased during EEG silence with all three challenges but was not different between the different bolus configurations. Laser Doppler blood flow showed no consistent relationship with bolus doses of propofol, although flows were higher during silence with 1% drug concentrations but this was not significant on post-hoc tests (Table III).

The inventor also observed that the dose requirement of intracarotid propofol required to produce 5 minutes of electrocerebral silence was a direct function of the bolus dose. Both the concentration and volume of the drug bolus had a significant effect on the dose requirements of intracarotid propofol.

TABLE II
Changes in Non-Hemodynamic Parameters with Different
Bolus Configurations of Intracarotid Propofol

| n = 8 | | Baseline | Drug | Recovery |
|---|---|---|---|---|
| Temperature (° C.) | 0.33% * 0.1 ml | 37.6 ± 1.1 | 37.6 ± 1.0 | 37.4 ± 1.0 |
| | 0.33% * 0.3 ml | 37.4 ± 1.1 | 37.4 ± 1.1 | 37.2 ± 1.0 |
| | 1.0% * 0.1 ml | 37.4 ± 0.9 | 37.3 ± 0.9 | 37.3 ± 0.9 |
| | 1.0% * 0.3 ml | 37.5 ± 0.8 | 37.4 ± 0.8 | 37.4 ± 0.8 |
| Respiratory rate | 0.33% * 0.1 ml | 36 ± 6 | 36 ± 6 | 36 ± 6 |
| (br.pm) | | | | |
| | 0.33% * 0.3 ml | 35 ± 3 | 35 ± 3 | 35 ± 3 |
| | 1.0% * 0.1 ml | 34 ± 4 | 34 ± 4 | 34 ± 4 |
| | 1.0% * 0.3 ml | 36 ± 6 | 37 ± 6 | 37 ± 6 |
| ETCO₂ (mm Hg) | 0.33% * 0.1 ml | 36 ± 3 | 35 ± 3 | 34 ± 3 |
| | 0.33% * 0.3 ml | 36 ± 2 | 35 ± 2 | 35 ± 2 |
| | 1.0% * 0.1 ml | 36 ± 3 | 35 ± 4 | 35 ± 3 |
| | 1.0% * 0.3 ml | 38 ± 5 | 37 ± 5 | 36 ± 5 |

Abbreviations:
ETCO₂: End-tidal carbon dioxide concentration,
br.pm: breaths per minute.
Symbols:
†significant post-hoc differences between challenges (P < 0.0083),
#significant post-hoc differences (P < 0.0167) between stages,
*from 0.33% * 0.1 ml,
@from 0.33% * 0.3 ml,
#from 1.0% * 0.1 ml.

Intracarotid bolus injections of drugs is frequently used for research, diagnostic and therapeutic purposes. Therefore, it is important to understand the kinetics of bolus injections (Dedrick R. L., Arterial drug infusion: pharmacokinetic problems and pitfalls. Journal of the National Cancer Institute 80:84-9, 1988). Bolus injections of drugs into the carotid artery can transiently achieve very high arterial drug concentrations. The kinetic of bolus drug administration is difficult to analyze due to the rapid changes in concentrations both in the arterial blood and the brain tissue. Jones et al observed that the tissue concentrations of benzodiazepines after bolus injection in rats were 5-25 times higher than that predicted by conventional protein binding parameters (Jones, et al., Brain uptake of benzodiazepines: effects of lipophilicity and plasma protein binding. J. Pharmacol. Exp. Ther. 245:816-22, 1988). The Jones reference offered several explanations of these observations, including rapid dissociation from binding proteins to permit enhanced uptake by the brain. While such factors could certainly have been involved, one possible biomechanical reason remained unexplored. It is possible that bolus injections of the drugs (0.15-0.2 ml as in the Jones reference) could have momentarily overwhelmed blood flow to deliver relatively undiluted drug to the brain in their rat model.

TABLE III
Changes in Systemic and Cerebrovascular Hemodynamic
Parameters with Different Bolus Configurations of
Intracarotid Propofol

| n = 9 | Challenge | Baseline | Drug | Recovery |
|---|---|---|---|---|
| Heart Rate | 0.33% * 0.1 ml | 260 ± 27 | 252 ± 24* | 252 ± 21 |
| (bpm) | | | | |
| | 0.33% * 0.3 ml | 259 ± 27 | 244 ± 27* | 249 ± 27* |
| | 1.0% * 0.1 ml | 252 ± 18 | 242 ± 24* | 245 ± 23 |
| | 1.0% * 0.3 ml | 255 ± 19 | 238 ± 26 | 244 ± 24 |
| MAP (mm Hg) | 0.33% * 0.1 ml | 90 ± 9 | 75 ± 13*# | 89 ± 9 |
| | 0.33% * 0.3 ml | 91 ± 9 | 72 ± 9*# | 88 ± 9 |
| | 1.0% * 0.1 ml | 89 ± 10 | 75 ± 9*# | 89 ± 8 |
| | 1.0% * 0.3 ml | 89 ± 9 | 67 ± 17*# | 86 ± 14 |
| ILD Flow (PU) | 0.33% * 0.1 ml | 210 ± 141 | 153 ± 91 | 151 ± 107* |
| | 0.33% * 0.3 ml | 210 ± 125 | 151 ± 103 | 155 ± 99 |
| | 1.0% * 0.1 ml | 236 ± 206 | 231 ± 207 | 195 ± 160 |
| | 1.0% * 0.3 ml | 228 ± 186 | 195 ± 150 | 164 ± 150* |
| CLD Flow | 0.33% * 0.1 ml | 190 ± 142 | 120 ± 90*# | 168 ± 137 |
| (PU) | | | | |
| | 0.33% * 0.3 ml | 199 ± 134 | 120 ± 86*# | 166 ± 135 |
| | 1.0% * 0.1 ml | 201 ± 161 | 135 ± 111* | 163 ± 125 |
| | 1.0% * 0.3 ml | 204 ± 161 | 140 ± 116* | 164 ± 138* |
| % Δ-ILD | 0.33% * 0.1 ml | 100 ± 0 | 77 ± 17* | 76 ± 24* |
| | 0.33% * 0.3 ml | 100 ± 0 | 71 ± 22* | 75 ± 22* |
| | 1.0% * 0.1 ml | 100 ± 0 | 94 ± 28 | 82 ± 14 |
| | 1.0% * 0.3 ml | 100 ± 0 | 88 ± 21 | 73 ± 14* |
| % Δ-CLD | 0.33% * 0.1 ml | 100 ± 0 | 64 ± 8* | 84 ± 15*@ |
| | 0.33% * 0.3 ml | 100 ± 0 | 61 ± 7* | 77 ± 14*@ |
| | 1.0% * 0.1 ml | 100 ± 0 | 71 ± 14* | 85 ± 11*@ |
| | 1.0% * 0.3 ml | 100 ± 0 | 69 ± 9* | 77 ± 11* |

Abbreviations:
bpm: beats per minute,
MAP: mean arterial pressure,
ILD: Ipsilateral laser Doppler,
PU: Perfusion Units,
CLD: Contralateral laser Doppler,
% Δ-ILD: %-change in ILD from baseline,
% Δ-CLD: %-change in CLD from baseline.
†significant post-hoc differences between challenges (P < 0.0083),
#significant post-hoc differences (P < 0.0167) between stages,
*from 0.33% * 0.1 ml,
@from 0.33% * 0.3 ml,
#from 1.0% * 0.1 ml.

Measurements suggest that in healthy rabbits the cerebral blood volume is 1.93 ml/100 g (Cenic, et al., Dynamic CT measurement of cerebral blood flow: a validation study. American Journal of Neuroradiology 20:63-73, 1999). Further assuming that the intracarotid injection irrigates 5 g of tissue, the inventor estimate that the total blood volume in a unilateral internal carotid irrigation is approximately 0.1 ml. However, in addition to cerebral tissue blood volume there is a comparable amount of blood in extracranial and intracranial arteries. Therefore, it is estimated that the total blood volume under the present experimental conditions was between 0.2 to 0.3 ml. Thus, injection of 0.1 and 0.3 ml under the conditions of the present studies would have certainly delivered relatively undiluted drug to the brain. In clinical settings, drugs are regularly administered at the rates of 1-10 ml/sec during cerebral angiography in humans. Such a rate of injection is sufficient to transiently overwhelm carotid blood flow and deliver relatively pure drug to the brain.

The primary advantage of bolus injection of drugs is the fairly consistent regional distribution of the drug (Castillo, et al., Cerebral amobarbital sodium distribution during Wada testing: utility of digital subtraction angiography and single-photon emission tomography. Neuroradiology 42:814-7, 2000). Bolus injections also deliver consistently high concentration and avoid regional variations in drug concentrations due to streaming (Lutz, et al., Mixing studies during intracarotid artery infusions in an in vitro model. J. Neurosurg. 64:277-83, 1986; Saris, et al., Carotid artery mixing with diastole-phased pulsed drug infusion. J. Neurosurg. 67:721-5, 1987). However, the disadvantage of bolus delivery is the limited uptake through the blood-brain barrier during the short time the bolus of drug has, as it transits within the brain. In theory, if the concentration of drug exceeds the maximum uptake by the brain in that transit period, then the extra amount of drug will simply overflow to the venous side. This would increase systemic side effects and decrease regional selectivity.

In the present study, the inventor observed that concentration of the drug played a subtly greater role than the volume of the bolus. With 0.33% concentration there was no difference in the total dose requirements between bolus volume of 0.1 and 0.3 ml. Within volume, a comparison revealed that there was a significant difference in dose requirements of the two concentrations of propofol, 1% and 0.33%. If the volume was relatively less important than concentration, then these results would suggest that the bolus volume was contained within the arterial dead space during the experiments. The relatively great dose with higher concentration of propofol might be due to a greater regional vasodilation with intra-arterial injection of the drug. The inventor observed a higher cerebral blood flow with 1% propofol than with 0.33%, although these changes were not significant. Such an increase blood flow will result in a loss of intracarotid drug.

There are several implications of this study. First, care must be exercised in interpreting dose-response studies with intracarotid bolus drug injection. Such studies may require a preliminary dose response experiments aimed to optimize drug delivery. Second, if intracarotid drugs are used for therapeutic purposes then, optimum bolus infusions regimes have to be described.

In conclusion, the inventor observed that the dose of propofol required to produce 5 minutes of EEG silence by intracarotid bolus injection was a function of the drug concentration and the volume injected. The study suggests that due care must be exercised in interpreting dose response of bolus injections of intracarotid drugs. Furthermore, when intracarotid drugs are injected for therapeutic purposes, bolus characteristics have to be optimized for maximal regional effect of the drug.

### Example 3

### Transient Flow Arrest Profoundly Increases the Duration of Electrocerebral Silence by Intracarotid Pentothal

For the present study, total recovery time was defined as time between the onset of electrocerebral silence after pentothal injection to electrocerebral activity comparable to baseline. Silence duration was the time elapsed between the injection of last bolus to the return of detectable electrocerebral activity, generally a burst-suppression pattern. Post-silence recovery time was described as the time between the onset of burst suppression to the return of electrocerebral activity comparable to the baseline. Hemodynamic and cerebral blood flow parameters for each drug were evaluated at three points of time: (i) baseline; (ii) during electrocerebral silence; and (iii) after recovery of electrocerebral activity.

Preliminary studies were undertaken to assess the optimum doses and cerebrovascular effects of drugs required to produce TCA. The preparation proved to be very tolerant to the effects of intravenous adenosine. Therefore, the inventor used an intravenous combination of esmolol 10 mg and 30 mg of adenosine, to produce severe systemic hypotension and flow arrest. This combination of drugs decreases the heart rate by 50-60%, and MAP and the laser Doppler flows to 20-30% of baseline values. Such a reduction in flow is sufficient to meet the criteria of flow arrest with laser Doppler measurements (Schmid-Elsaesser, et al., A critical reevaluation of the intraluminal thread model of focal cerebral ischemia: evidence of inadvertent premature reperfusion and subarachnoid hemorrhage in rats by laser-Doppler flowmetry. Stroke 29:2162-70, 1998).

The definitive study required comparisons between the effects of intracarotid pentothal with normal CBF and during flow arrest in the brain. There was a possibility that severe hypotension with the concurrent use of intra-arterial pentothal could injure the preparation. Due to the possibility of injury the inventor did not randomize the two interventions, but assessed the effects of pentothal before and after the hypotensive challenge. This helped assess the time-dependent, post-arrest, and residual drug effects on the preparation.

After baseline measurements of physiological parameters under normocapnic conditions were obtained, the animal received a standard injection of 3 mg of intracarotid pentothal. The loading dose of 1% pentothal is about 0.3±0.1 ml (Joshi, et al., Electrocerebral silence by intracarotid anesthetics does not affect early hyperemia after transient cerebral ischemia in rabbits. Anesth. Analg. 98:1454-9, 2004). Thus, a volume dose of 0.5 ml of 1% pentothal, assures adequate drug delivery to the brain to illicit consistent drug effects. Considering that 0.2 ml will remain in the dead space of the catheter and the stopcock, an effective dose of 3 mg was actually delivered to the cerebral circulation. Systemic hemodynamic effects, cerebrovascular, and the electrocerebral activity effects of the drugs were continuously recorded. The preparation was allowed to recover for 45 min. In the next stage, intravenous esmolol and adenosine were injected intravenously while pentothal was injected through the carotid artery. Electro-physiological and hemodynamic parameters were assessed thereafter. The preparation was then allowed to recover for another 45 min. After this, a repeat bolus of pentothal was injected via the intracarotid route.

The data is presented as mean±standard deviation. The hemodynamic and laser Doppler flow data recorded at three time points (baseline, silence and recovery) were normalized to baseline value. A P value of <0.05 was considered significant between the three challenges (pentothal-1, pentothal+arrest, and pentothal-2, ANOVA factorial). A P<0.0167 was considered significant between the three stages of each challenge (baseline, drug and recovery). All of which were evaluated by ANOVA repeated measures with Bonferroni Dunn test for multiple comparisons.

Discussed below are results obtained by the inventor in connection with the experiments of Examples 1 and 3:

Preliminary studies evaluated the effects of severe hypotension by esmolol and adenosine on electrophysiological and hemodynamic parameters. The preliminary studies were conducted on 4 animals to evaluate the cerebrovascular and electrophysiological effects of severe systemic hypotension in the absence of intracarotid drugs. As shown in Table IV, injection of adenosine 30 mg and esmolol 10 mg decreased the MAP to 94±11 to 26±2 mm Hg, P<0.0001. During hypotension, the heart rate decreased from 257±20 to 132±26 beats/min, n=4, P=0.0003. The electrocerebral activity was attenuated during hypotension in all four animals immediately after injection of esmolol and adenosine. Blood flow declined from 147±78 to 47±29 PU, P=0.0083, i.e., to 20-30% of baseline values during hypotension. MAP and the HR returned to near baseline values within 3±1 minutes of drug injection. No inotropic support was required during recovery.

Definitive study was conducted on 10 animals. In one animal, the electrocerebral activity did not return to baseline amplitude and morphology after the arrest. Only data from the other nine animals were included in the final analysis. The definitive study involved three repeat challenges of drugs, (i) pentothal-1, (ii) pentothal+arrest, and (iii) pentothal-2, respectively. Intracarotid injection of pentothal prior to the flow arrest (pentothal-1) produced 45±5 seconds of electrocerebral silence (Table V). Post arrest injection of pentothal (pentothal-2) produced 67±27 seconds of electrocerebral silence that was not significantly different from pentothal-1 (n=9, P=0.132). The total recovery time was significantly prolonged during pentothal+arrest (291±60 seconds) but was comparable between pentothal-1 (126±29 seconds) and pentothal-2 (161±71 seconds). However, the time between the post-silence recovery was similar in the three groups pentothal-1, pentothal+arrest, and pentothal-2 (81±27, 85±27, and 94±55 seconds, respectively). Injection of pentothal 3 mg during flow arrest produced 206±46 seconds of silence that was significantly different from pentothal-1 (46±5 seconds, P<0.0001) and pentothal-2 (67±27 seconds, P<0.0001). The MAP, HR, ETCO2 and laser Doppler flows were significantly lower during pentothal+arrest (Table VI and VII). Ipsilateral laser Doppler flow were 130±59 to 33±11 P.U., i.e., to <20-30% of baseline values. Cerebral and systemic hemodynamic parameters were comparable between the two pentothal challenges.

Although TCA has been extensively used during endovascular surgery, this is the first study to evaluate the possibility of using flow arrest as a tool to enhance delivery of drugs to the brain. The inventor observed that intracarotid injection of pentothal during flow arrest, significantly prolonged the duration of electrocerebral silence, although post-silence recovery time was similar with all the three challenges. These results suggest that modulation of blood flow to the brain is a critical factor in influencing the efficacy of intra-arterial drugs. The data further suggest that the increase in duration of electrocerebral silence is due to higher concentrations of drug in the brain, and not due to slow rate of drug washout once flow is restored.

TABLE IV
Preliminary Studies Showing The Effect Of Flow Arrest
On Hemodynamic And Cerebral Blood Flow Parameters

| n = 4 | Baseline | Arrest | Recovery |
|---|---|---|---|
| Heart Rate (bpm) | 257 ± 20 | 133 ± 26# | 235 ± 25 |
| Temperature (° C.) | 36.4 ± .7 | 36.3 ± .7 | 36.4 ± .6 |
| ETCO₂ (mm Hg) | 38 ± 2 | 31 ± 7# | 37 ± 3 |
| Respiratory rate | 32 ± 3 | 32 ± 3 | 32 ± 3 |
| (br.pm) | | | |
| MAP (mm Hg) | 94 ± 11 | 27 ± 2# | 97 ± 8 |
| ILD Flow (PU) | 147.14 ± 78.05 | 46.84 ± 28.75# | 170.34 ± 92.24 |
| CLD Flow (PU) | 118.67 ± 58.65 | 51.51 ± 28.81# | 124.88 ± 61.19 |
| % Δ-ILD | 100 | 31.03 ± 5.64# | 114.23 ± 10.86 |
| % Δ-CLD | 100 | 41.64 ± 5.76# | 105.89 ± 10.42 |

Abbreviations:
bpm: beats per minute,
ETCO₂: End-tidal carbon dioxide concentration,
br.pm: breaths per minute,
MAP: mean arterial pressure,
ILD: Ipsilateral laser Doppler,
PU: Perfusion Units,
CLD: Contralateral laser Doppler,
% Δ-ILD: %-change in ILD from baseline,
% Δ-CLD: %-change in CLD from baseline.
#significant post-hoc differences between stages (P < 0.0167).

TABLE V
The Effect of Intracarotid Pentothal on the Duration
of EEG Parameters

| N = 9 | Pentothal-1 | Pentothal + Arrest | Pentothal-2 |
|---|---|---|---|
| Silence Duration(s) | 45 ± 5 | 206 ± 46* | 67 ± 27 |
| Total Recovery | 126 ± 29 | 291 ± 60* | 161 ± 71 |
| Time(s) | | | |
| Post-silence recovery | 81 ± 27 | 85 ± 27 | 94 ± 55 |
| time (s) | | | |

*significant differences between challenges (P < 0.05)

Adenosine and esmolol are both exceedingly short acting drugs. A combination of these drugs was sufficient to produce a severe reduction in laser Doppler flow to 20-30% of baseline values, which is sufficient to meet the criteria of flow arrest by laser Doppler measurements. However, the use of such high doses of the drug made randomization difficult. Rather than randomize the drugs, the inventor tested the response to pentothal before and after the pharmacological flow arrest. By using two control challenges the inventor could assess changes in the preparation due to time, possible ischemic injury and the residual effects of systemic drugs. The results of pentothal-1 and pentothal-2 challenges were fairly similar (Table VI and VII), which suggest a minimal residual effect of flow arrest on electrocerebral response to intracarotid pentothal.

TABLE VI
Changes In Non-Hemodynamic Parameters During The
Three Pentothal Challenges

| N = 9 | | Baseline | Drug | Recovery |
|---|---|---|---|---|
| Temperature | Pentothal-1 | 36.6 ± 1.0 | 36.5 ± 1.0 | 36.5 ± 1.0 |
| (° C.) | Pentothal + Arrest | 36.4 ± 0.9 | 36.4 ± 0.8 | 36.3 ± 0.8 |
| | Pentothal-2 | 36.4 ± 0.8 | 36.5 ± 0.7 | 36.4 ± 0.7 |
| | Pentothal-1 | 30 ± 5 | 30 ± 5 | 31 ± 5 |
| | Pentothal-1 | 30 ± 5 | 30 ± 5 | 31 ± 5 |
| Respiratory | Pentothal + 2Arrest | 31 ± 4 | 31 ± 4 | 31 ± 4 |
| rate | | | | |
| ( [symbol] ) (mm Hg) | Pentothal-1 | 37 ± 2 | 37 ± 2* | 36 ± 2 |
| | Pentothal + Arrest | 37 ± 3 | 31 ± 3# | 34 ± 4 |
| | Pentothal-2 | 37 ± 3 | 37 ± 4* | 36 ± 4 |

Abbreviations:
ETCO₂: End-tidal carbon dioxide concentration,
br.pm: breaths per minute.
*significant post hoc differences between challenges (P < 0.05),
#significant post-hoc differences between stages (P < 0.0167).

TABLE VII
Changes In Systemic And Cerebrovascular Hemodynamic
Parameters During Intracarotid Injection Of Pentothal

| N = 9 | Challenge | Baseline | Drug | Recovery |
|---|---|---|---|---|
| Heart Rate | Pentothal-1 | 262 ± 42 | 262 ± 36* | 241 ± 29 |
| (bpm) | Pentothal + Arrest | 265 ± 21 | 125 ± 19# | 258 ± 44 |
| | Pentothal-2 | 261 ± 23 | 254 ± 20* | 268 ± 22 |
| MAP (mm Hg) | Pentothal-1 | 101 ± 14 | 94 ± 17* | 102 ± 7 |
| | Pentothal + Arrest | 98 ± 11 | 26 ± 2# | 91 ± 14 |
| | Pentothal-2 | 96 ± 13 | 91 ± 21* | 99 ± 11 |
| ILD Flow (PU) | Pentothal-1 | 141 ± 73 | 147 ± 83* | 140 ± 66 |
| | Pentothal + Arrest | 130 ± 59 | 33 ± 11# | 161 ± 81 |
| | Pentothal-2 | 143 ± 76 | 140 ± 55* | 129 ± 68 |
| CLD Flow | Pentothal-1 | 106 ± 45 | 111 ± 50* | 93 ± 23 |
| (PU) | Pentothal + Arrest | 114 ± 48 | 41 ± 21# | 106 ± 34 |
| | Pentothal-2 | 111 ± 53 | 118 ± 41* | 89 ± 38 |
| % Δ-ILD | Pentothal-1 | 100 ± 0 | 106 ± 51* | 101 ± 29 |
| | Pentothal + Arrest | 100 ± 0 | 27 ± 8# | 124 ± 33 |
| | Pentothal-2 | 100 ± 0 | 104 ± 30* | 92 ± 38 |
| % Δ-CLD | Pentothal-1 | 100 ± 0 | 106 ± 38* | 94 ± 19 |
| | Pentothal + Arrest | 100 ± 0 | 36 ± 9# | 100 ± 32 |
| | Pentothal-2 | 100 ± 0 | 112 ± 26* | 83 ± 22 |

Abbreviations:
bpm: beats per minute,
MAP: mean arterial pressure,
ILD: Ipsilateral laser Doppler,
PU: Perfusion Units,
CLD: Contralateral laser Doppler,
% Δ-ILD: %-change in ILD from baseline,
% Δ-CLD: %-change in CLD from baseline.
*significant post hoc differences between challenges (P < 0.05),
#significant post-hoc differences between stages (P < 0.0167).

In clinical practice, intra-arterial drugs have been used effectively during the treatment of cerebral vasospasm, a condition of low cerebral blood flow (Oskouian, et al., Multimodal quantitation of the effects of endovascular therapy for vasospasm on cerebral blood flow, transcranial doppler ultrasonographic velocities, and cerebral artery diameters. Neurosurgery 51:30-41, 2002). However, intra-arterial delivery has been less efficacious in other settings, such as in the treatment of brain tumors (Oldfield, et al., Reduced systemic drug exposure by combining intra-arterial chemotherapy with hemoperfusion of regional venous drainage. J Neurosurg. 63:726-32, 1985). A number of factors, such as inadequate penetration of blood brain barrier by drugs, may explain the therapeutic failures of intra-arterial chemotherapy. However, no attempt was made in the past to modulate blood flow to enhance intra-arterial drug delivery to the brain, which is a key determinant of drug delivery to the brain. By using computer simulations, Dedrick R. L. reported that intra-arterial drugs was efficacious in three specific situations: drugs with high systemic clearance, drugs with selective brain uptake, and drugs administered in areas of low regional blood flow (Dedrick R. L., Arterial drug infusion: pharmacokinetic problems and pitfalls. Journal of the National Cancer Institute 80:84-9, 1988). It is to be noted that when anesthetic drugs are administered intravenously, augmentation of CBF enhances drug delivery to the brain (Upton, et al., The effect of altered cerebral blood flow on the cerebral kinetics of thiopental and propofol in sheep. Anesthesiology 93:1085-94, 2000). The converse seems to be true with intra-arterial drug delivery.

There are two outstanding concerns in employing flow arrest to the brain. The first concern is the possibility of ischemic cerebral injury and the second concern is the occurrence reactive hyperemia. In the present model, the duration of flow arrest was very transient <20 seconds and the flows rapidly returned to near baseline values within 1 min of hypotension. The inventor observed transient attenuation of electrocerebral activity during flow arrest that in the absence of pentothal, and the electrocerebral activity rapidly (<45 seconds) returned to baseline amplitude and morphology. These data suggest that the magnitude of reduction of flow in the present model was not associated with injury. If flow arrest is clinically used, the duration of flow arrest has to be sufficiently short so as to avoid any ischemic injury. The second hazard of flow arrest is the reactive hyperemia. While the clinical impact of post-ischemic reactive hyperemia can be debated, such an increase in flow will enhance drug elimination from the brain. The inventor did not observe a significant increase in laser Doppler flow after transient flow arrest during the preliminary studies. Previously, the inventor has observed significant increases in laser Doppler flows occur in the experimental model when ischemia last for about 10 min (Joshi, et al., Electrocerebral silence by intracarotid anesthetics does not affect early hyperemia after transient cerebral ischemia in rabbits. Anesth. Analg. 98:1454-9, 2004). It seems that transient ischemia of <20 seconds duration does not result in significant hyperemia.

There are few studies that have assessed electrocerebral activity changes as a function of the concentrations of pentothal in the brain. In sheep, electrocerebral silence is evident when tissue concentration of pentothal that produce electrocerebral silence is about 0.50 mg/dl (Mather, et al., Electroencephalographic effects of thiopentone and its enantiomers in the rat. Life Sciences 66:105-14, 2000). However, there are a number of studies that correlate electro-encephalographic changes and arterial pentothal concentrations. In the present study, the administration of pentothal during flow arrest prolonged the duration of electrocerebral silence, however, the recovery of electroencephalographic morphology after the end of silence was not affected by flow arrest. Recovery from electrocerebral silence will be a function of peak tissue concentrations, redistributive half-life of pentothal, and the regional blood flow. Relative to the prolongation in the duration of electrocerebral silence (3-5 folds) with pentothal+arrest vs. pentothal-1 and 2, the ipsilateral CBF remained low during recovery, and was comparable with the three challenges. Thus, the decrease in blood flow could not have explained the increased duration of electrocerebral silence. The results of the present study suggest that the prolongation of electrocerebral silence by intracarotid pentothal during flow arrest, was primarily due to a higher tissue concentrations.

The present study demonstrates that the administration of intracarotid pentothal during flow arrest increases the duration of drug effect, which indicates that modulation of blood flow might be an important tool in enhancing intra-arterial drug delivery to the brain.

### Example 4

### The Effects of Ventilation on the Dose Requirements of Intracarotid Propofol for Electrocerebral Silence in Rabbits

During the definitive study baseline measurements of physiological parameters were obtained under normocapnic conditions. Animals were then randomly subjected to (i) normocapnic ventilation with an ETCO₂ of 30-35 mm Hg, (ii) hyperventilation, ETCO₂ of 20-25 mm Hg, and (iii) hypoventilation, ETCO₂ of 45-50 mm Hg. Minute ventilation was altered by changing the respiratory rate. Ventilation was maintained for 5 minutes before intracarotid propofol was injected. To determine the loading dose, propofol (1% Diprivan, 0.1 ml) was injected every 10 seconds, until electrocerebral silence was evident for at least 10 seconds. Thereafter, repeat doses of the drug (maintenance dose) were administered whenever electrocerebral activity was evident or when bursts of electrocerebral activity returned. The silence was maintained for 10 minutes. Then, the preparation was allowed to recover without altering the ventilation. The total dose anesthetic drug required electrocerebral silence was the sum of loading and maintenance doses. Once electrocerebral activity, CBF, and MAP had returned to pre-drug levels, the ventilation was altered for the next ventilatory challenge.

The data are presented as mean±standard deviation. The hemodynamic and laser Doppler flow data recorded at the three time points (baseline, silence and recovery) was normalized to baseline value and analyzed by repeated measures ANOVA. Bonferroni-Dunn post hoc test to correct for multiple comparisons was undertaken to determine significance. A P value of <0.0167 was considered as significant.

Discussed below are results obtained by the inventor in connection with the experiments of Examples 1 and 4:

The study was conducted on 10 New Zealand white rabbits weighing 1.5±0.5 kg. Satisfactory data could be collected from 9 of the 10 animals. There was a failure to correctly isolate the internal carotid artery in one animal. Thus, 27 data points were available from 9 animals. The mean ETCO₂ was significantly different during normocapnia, hypocapnia and hypercapnia, 36±1, 24±3, and 47±3 mm Hg, respectively, n=9, P<0.0001. The temperature remained constant during the study, Table VIII. Compared to normocapnia and hypercapnia, hypocapnia was associated with hypotension and tachycardia. Hypercapnia was associated with a significant increase in CBF. Despite significant differences in ETCO₂, there was no difference in blood flow during hypocapnia and normocapnia. Despite a significant increase in respiratory rate, and a decrease in ETCO₂, CBF and CVR did not decrease during hypocapnia.

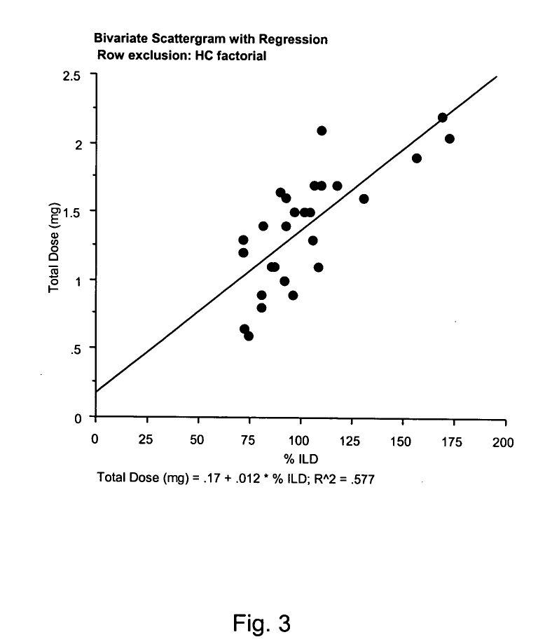

The dose requirements of intracarotid propofol were significantly affected by the change in ventilation. The total dose of the drug was the highest for hypercapnia (1.8±0.3 mg) compared to both hypocapnia (1.0±0.3 mg) and normocapnia (1.4±0.3 mg), n=27, P<0.0001 from hypocapnia, and 0.0062 from normocapnia (Table IX). There was a significant correlation between the total, loading and maintenance doses and the %-change in blood flow from baseline, (Table X and FIG. 3).

TABLE VIII
Changes in Systemic Parameters During Hypercapnic,
Hypocapnic and Normocapnic Ventilation

| | Ventilatory | | | |
|---|---|---|---|---|
| N = 9 | state | Pre-drug | Drug | Recovery |
| Temperature | Hypercapnia | 36 ± 1 | 36 ± 1 | 36 ± 1 |
| (° C.) | Hypocapnia | 36 ± 1 | 36 ± 1 | 36 ± 1 |
| | Normocapnia | 37 ± 1 | 37 ± 1 | 36 ± 1 |
| Respiratory | Hypercapnia | 24 ± 5* | 24 ± 5* | 25 ± 5* |
| rate (br.pm) | Hypocapnia | 74 ± 7* | 74 ± 7* | 74 ± 7* |
| | Normocapnia | 44 ± 6* | 45 ± 6* | 44 ± 6* |
| Heart Rate | Hypercapnia | 219 ± 34 | 218 ± 30 | 221 ± 27 |
| (bpm) | Hypocapnia | 265 ± 24* | 254 ± 23*# | 252 ± 27*# |
| | Normocapnia | 264 ± 29* | 262 ± 21* | 254 ± 32* |
| MAP (mm Hg) | Hypercapnia | 88 ± 14 | 79 ± 17# | 89 ± 13 |
| | Hypocapnia | 83 ± 18 | 72 ± 19*# | 82 ± 24 |
| | Normocapnia | 93 ± 12 | 85 ± 16# | 91 ± 13 |
| ETCO₂ (mm | Hypercapnia | 47 ± 3* | 49 ± 3* | 49 ± 2* |
| Hg) | Hypocapnia | 24 ± 3* | 23 ± 3* | 22 ± 2* |
| | Normocapnia | 36 ± 1* | 36 ± 2* | 34 ± 3* |

Abbreviations:
ETCO₂: End-tidal carbon dioxide concentration,
br.pm: breaths per minute.
*significant post hoc differences between ventilatory states (P < 0.0167),
#significant post-hoc differences between stages of each drug challenge (P < 0.0167).

TABLE IX
The Effect of Ventilation on Dose Requirements
of Intracarotid Propofol

| N = 9 | Hypercapnia | Hypocapnia | Normocapnia |
|---|---|---|---|
| Total Dose (mg) | 1.8 ± 0.3 | 1.0 ± 0.3* | 1.4 ± 0.3# |
| Loading Dose (mg) | 0.6 ± 0.2 | 0.3 ± 0.1* | 0.4 ± 0.1# |
| Maintenance Dose (mg) | 1.2 ± 0.3 | 0.7 ± 0.3* | 1.0 ± 0.3 |

Symbols significant differences between ventilatory states (P < 0.0167):
*between hypercapnia and hypocapnia and between
#hypercapnia and normocapnia.

TABLE X
Changes In Cerebrovascular Parameters During Hypercapnic,
Hypocapnic and Normocapnic Ventilations

| | Ventilatory | | | |
|---|---|---|---|---|
| N = 9 | state | Pre-drug | Drug | Recovery |
| ILD Flow | Hypercapnia | 201 ± 71 | 175 ± 83 | 146 ± 68 |
| (PU) | Hypocapnia | 141 ± 65 | 113 ± 51 | 104 ± 43 |
| | Normocapnia | 142 ± 76 | 130 ± 67 | 110 ± 62 |
| CLD Flow | Hypercapnia | 250 ± 164* | 194 ± 140*# | 174 ± 126# |
| (PU) | Hypocapnia | 173 ± 164* | 124 ± 95* | 129 ± 104 |
| | Normocapnia | 188 ± 130* | 160 ± 107* | 148 ± 102# |
| % Δ-ILD | Hypercapnia | 157 ± 54 | 129 ± 29 | 107 ± 20# |
| | Hypocapnia | 104 ± 22 | 84 ± 12 | 81 ± 23# |
| | Normocapnia | 101 ± 19 | 93 ± 14 | 82 ± 6# |
| % Δ-CLD | Hypercapnia | 176 ± 50 | 133 ± 40 | 113 ± 22 |
| | Hypocapnia | 101 ± 47 | 77 ± 15 | 79 ± 15 |
| | Normocapnia | 105 ± 27 | 86 ± 19# | 82 ± 19# |

Abbreviations:
bpm: beats per minute,
MAP: mean arterial pressure,
ILD: Ipsilateral laser Doppler,
PU: Perfusion Units,
CLD: Contralateral laser Doppler,
% Δ-ILD: %-change in ILD from baseline,
% Δ-CLD: %-change in CLD from baseline.
*significant post hoc differences between ventilatory states (P < 0.0167),
#significant post-hoc differences between stages of each drug challenge (P < 0.0167).

The results of this study reveal that the dose requirements of intra-arterial propofol required to produce 10 minutes of electrocerebral silence is significantly affected by ventilation. Decreased minute ventilation, increased ETCO₂ and CBF was associated with the increased dose requirements of intracarotid propofol. This study supports the concept that an increase in CBF adversely affects the dose requirements of intra-arterial drugs (Fenstermacher and Cowles, Theoretic limitations of intracarotid infusions in brain tumor chemotherapy. Cancer Treat. Rep. 61:519-26, 1977; Dedrick R. L., Arterial drug infusion: pharmacokinetic problems and pitfalls. J. NCI 80:84-9, 1988). Furthermore, this study reveals that it is feasible to affect the dose requirement of some intracarotid drugs by altering the minute ventilation.

It is well known that the dose requirement for intravenous anesthetics decreased with the increase in CBF (Upton, et al., The effect of altered cerebral blood flow on the cerebral kinetics of thiopental and propofol in sheep. Anesthesiology 93:1085-94, 2000; Upton, et al., Cardiac output is a determinant of the initial concentrations of propofol after short-infusion administration. Anesth. Analg. 89:545-52, 1999). This is due to a greater proportion of the systemically administered drug being delivered to the brain. However, during intracarotid delivery, the delivery of the drug to the brain is not the rate-limiting step. With intra-arterial delivery, the uptake of the drug by the brain becomes the rate-limiting step. The factors could alter the uptake included the ability of the drug to penetrate the blood brain barrier and CBF. The higher CBF, the greater the dilution of the drug, the shorter the transit time, and the more rapid washout. Thus, increased CBF may limit drug uptake by the brain with intra-arterial delivery.

Few studies have addressed the kinetics of intracarotid bolus drug injections (Reichenthal. et al., The feasibility of low dose barbiturate administration by intra-carotid infusion to achieve EEG burst suppression—a preliminary report. Neurochirurgia (Stuttg) 31:50-3, 1988; Wang, et al., Comparison of Intracarotid and Intravenous Propofol for Electrocerebral Silence in Rabbits. Anesthesiology 99:904-10, 2003). Jones, et al., in a murine model, observed 5-25 fold higher benzodiazepine concentrations in the brain than those predicted by protein binding of the drugs (Jones, et al., Brain uptake of benzodiazepines: effects of lipophilicity and plasma protein binding. J. Pharmacol. Exp. Ther. 245:816-22, 1988). Propofol is a highly lipid-soluble drug with an octanol:water partition coefficient of 6761. It also is highly non-ionized and is highly protein-bound (98%). The high protein-binding of propofol would decrease its uptake by the brain and could explain an prolonged equilibrium time with the brain and blood (3-5 minutes, based on intravenous infusions) (Ludbrook, et al., Brain and blood concentrations of propofol after rapid intravenous injection in sheep, and their relationships to cerebral effects. AAIC 24:445-52, 1996; Ludbrook, et al., The effect of rate of administration on brain concentrations of propofol in sheep. Anesth. Analg. 86:1301-6, 1998; Upton and Ludbrook, A model of the kinetics and dynamics of induction of anaesthesia in sheep: variable estimation for thiopental and comparison with propofol. Br. J. Anaesth. 82:890-9, 1999). However, during bolus injections protein binding might be a less significant factor. It has been estimated that the blood volume in the rabbit brain is 1.89 ml/100 g (Cenic, et al., Dynamic CT measurement of cerebral blood flow: a validation study. Am. J. Neuroradiol. 20:63-73, 1999). Assuming the internal carotid artery irrigates 5 g of brain tissue the effective blood volume will be approximately 0.1 ml, equivalent to the bolus volume of the injected drug. Thus, during the present experiments relatively concentrated drug was being delivered to the brain. The results of the present study suggest that, during bolus drug injections, CBF is transiently overwhelmed and that relatively pure free drug is delivered to the brain. In human setting, intra-arterial drug boluses are often given in a rate of 1-10 ml/seconds during angiographic procedures when the estimated carotid blood flow is about ≈3 ml/seconds.

It is difficult to investigate the kinetics of intracarotid bolus drug delivery. Techniques like microdialysis are challenging because of low volume yield of microdialysate, which is ≈2 μl/min. Such a low yield may be insufficient to detect changes in drug concentration when delivered over 1-2 seconds. The inventor has used electrocerebral activity changes as a surrogate measure of tissue concentration. Despite limitation imposed by acute tolerance and hysterises, the results demonstrates that the model used in the present study provides a useful insight into the kinetics of intracarotid drug delivery (Ludbrook, et al., Brain and blood concentrations of propofol after rapid intravenous injection in sheep, and their relationships to cerebral effects. AAIC 24:445-52, 1996).

The inventor concluded that doses of intracarotid propofol required to produce 10 minutes of electrocerebral silence, are significantly affected by the ventilation. Furthermore, the dose-requirements of intracarotid propofol are directly related to the changes in CBF. The study reveals that manipulation of ventilation might be an effective tool in modulating intra-arterial delivery of drugs to the brain.

### Example 5

### Transient Flow Arrest Profoundly Increases the Duration of Electrocerebral Silence by Intracarotid Propofol

In the manner described in Example 3 the inventor assessed the significance of flow arrest vs. change in concentration of another drug, propofol. The study was conducted on eight New Zealand rabbits that were subjected to six drug challenges. They all received propofol 1 or 3 mg before arrest, 1 or 3 mg during flow arrest and 1 or 3 mg after arrest. The flow arrest was produced by intravenous injection of esmolol (10 mg) and adenosine (30 mg).

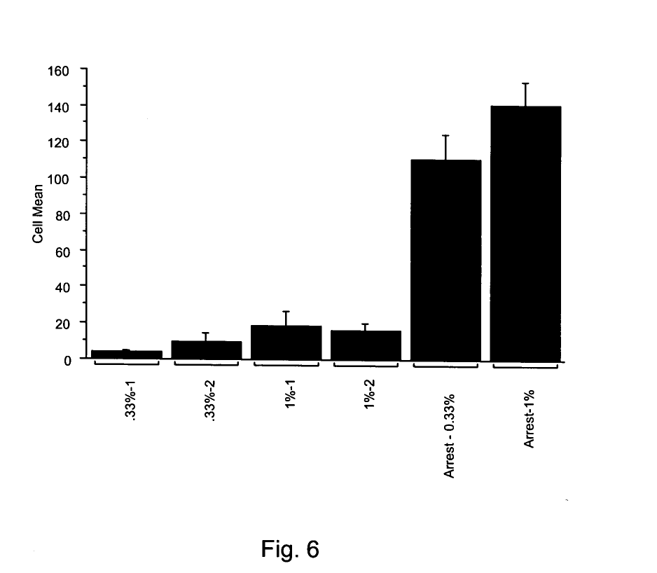

FIG. 6 depicts the effect of different concentration of propofol (0.33% and 1%) on the duration of electrocerebral silence before (0.33%-1 and 1%-1), during (Arrest-0.33% and Arrest-1%) and after (0.33%-2 and 1%-2). Severe hypotesion lasted 3-5 minutes and flow arrest (to ≈25% of baseline) lasted for 20 seconds. Changing concentration increased the duration of EEG silence by 2-4 folds (P=0.02, NS on post-hoc testing) while flow arrest increased the duration of EEG silence 15-20 folds (n=8, P<0.0001).

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A steerable device comprising or which comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

2. The steerable device of claim 1, wherein the proximal double-lumen assembly is about 4-5.5 French in diameter for the first 80-100 cm.

3. The steerable device of claim 2, wherein the diameter of the proximal double-lumen assembly is gradually narrowed to about 2-3 French in the distal end.

4. The steerable device of claim 1, wherein the micro-catheter is extended beyond the distal end of the balloon.

5. The steerable device of claim 4, wherein the micro-catheter is extended 1-10 cm beyond the distal end of the balloon.

6. The steerable device of claim 1, wherein the micro-catheter is about 1-1.5 French in diameter.

7. The steerable device of claim 1, wherein the balloon is about 1-1.5 cm in length.

8. The steerable device of claim 1, wherein the catheter is made of a material which renders the steerable device strong enough to withstand repeated inflation and deflation of the balloon, flexible enough to negotiate the curve of blood vessels, and having low frictional resistance and thrombogenic potential.

9. The steerable device of claim 1, wherein the steerable device is made of a material with high tensile strength.

10. The steerable device of claim 9, wherein the high tensile strength material is selected from the group consisting of Teflon, nylon, polyurethane, and polyethylene.

11. The steerable device of claim 1, wherein the steerable device has a surface coating.

12. The steerable device of claim 11, wherein the surface coating is hydrophilic.

13. A drug delivery system comprising a steerable device and a balloon drive, wherein the steerable device comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

14. The drug delivery system of claim 13, wherein the proximal double-lumen assembly is about 4-5 French in diameter for the first 80-100 cm.

15. The drug delivery system of claim 14, wherein the diameter of the proximal double-lumen assembly is gradually narrowed to about 2-3 French in the distal end.

16. The drug delivery system of claim 13, wherein the micro-catheter is extended beyond the distal end of the balloon.

17. The drug delivery system of claim 16, wherein the micro-catheter is extended 1-10 cm beyond the distal end of the balloon.

18. The drug delivery system of claim 13, wherein the micro-catheter is about 1-1.5 French in diameter.

19. The drug delivery system of claim 13, wherein the balloon is about 1-1.5 cm in length.

20. The drug delivery system of claim 13, wherein the steerable device is made of a material which renders the steerable device strong enough to withstand repeated inflation and deflation of the balloon, flexible enough to negotiate the curve of blood vessels, and having low frictional resistance and thrombogenic potential.

21. The drug delivery system of claim 13, wherein the steerable device is made of a material with high tensile strength.

22. The drug delivery system of claim 21, wherein the high tensile strength material is selected from the group consisting of Teflon, nylon, polyurethane and polyethylene.

23. The drug delivery system of claim 13, wherein the steerable device has a surface coating.

24. The drug delivery system of claim 23, wherein the surface coating is hydrophilic.

25. The drug delivery system of claim 13, wherein the balloon drive controls the inflation and deflation of the balloon.

26. The drug delivery system of claim 13, further comprising a computerized device, wherein the computerized device controls the balloon drive.

27. The drug delivery system of claim 26, wherein the primary parameters used by the computerized device to control the balloon drive are the frequency, duration and volume of inflation/deflation.

28. The drug delivery system of claim 13, wherein the balloon is inflated by a radio-opaque low viscosity fluid.

29. The drug delivery system of claim 13, wherein the balloon can be inflated or deflated in about 1 second.

30. A method for the localized delivery of at least one agent to a target location within a subject, comprising the steps of:
(1) partially or completely arresting blood flow to the target location for a short period of time;
(2) delivering the at least one agent in bolus to the target location; and
(3) partially or completely restoring blood flow to the target tissue,
(4) wherein the blood flow is arrested by occluding the artery to the target tissue.

31. The method of claim 30, wherein the steps of (1)-(3) are repeated at least once.

32. The method of claim 30, wherein the blood flow is arrested by inflating a balloon and restored by deflating the balloon.

33. The method of claim 32, wherein the balloon is inflated or deflated in about 1 second.

34. The method of claim 32, wherein a balloon drive controls the inflation and deflation of the balloon.

35. The method of claim 34, wherein a computerized device controls the balloon drive.

36. The method of claim 32, wherein the duration of the balloon inflation is about 10-50 seconds.

37. The method of claim 30, wherein the ventilation of the subject is increased.

38. The method of claim 30, wherein the at least one agent is delivered using a steerable device.

39. The method of claim 38, wherein the steerable device comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

40. The method of claim 30, the at least one agent is a therapeutic or diagnostic agent.

41. The method of claim 40, wherein the therapeutic agent is selected from the group consisting of an agent for treating brain-related disorders, a chemotherapeutic agent, and a gene-therapy agent.

42. The method of claim 30, wherein the target location is in or close to a tissue in the subject, wherein the tissue has a pathological condition.

43. The method of claim 42, wherein the target location is the artery in or near brain, a tumor, or a tissue in need of gene-therapy.

44. The method of claim 30, wherein the subject is a mammal.

45. The method of claim 30, wherein the subject is a human.

46. A method for the localized delivery of at least one agent to a target location within a subject, comprising the steps of:
(1) providing a drug delivery system comprises a steerable device and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated;
(2) incorporating the at least one agent into the drug delivery system; and
(3) delivering the at least one agent to the target location.

47. The method of claim 46, wherein the micro-catheter is extended beyond the distal end of the balloon.

48. The method of claim 46, wherein the steerable device is made of a material with high tensile strength.

49. The method of claim 48, wherein the high tensile strength material is selected from the group consisting of Teflon, nylon, polyurethane, and polyethylene.

50. The method of claim 46, wherein the steerable device has a surface coating.

51. The method of claim 50, wherein the surface coating is hydrophilic.

52. The method of claim 46, wherein the balloon drive controls the inflation and deflation of the balloon.

53. The method of claim 46, further comprising a computerized device, wherein the computerized device controls the balloon drive.

54. The method of claim 53, wherein the primary parameters used by the computerized device to control the balloon drive are the frequency, duration and volume of inflation/deflation.

55. The method of claim 46, wherein the balloon is inflated by a radio-opaque low viscosity fluid.

56. The method of claim 46, wherein the balloon can be inflated or deflated in about 1 second.

57. The method of claim 46, wherein the duration of the balloon inflation is about 10-50 seconds.

58. The method of claim 46, wherein the balloon is rapidly inflated and deflated at least once.

59. The method of claim 46, wherein the balloon inflation partially or completely arrests the blood flow to the target location.

60. The method of claim 46, wherein the ventilation of the subject is increased.

61. The method of claim 46, the at least one agent is a therapeutic or diagnostic agent.

62. The method of claim 61, wherein the therapeutic agent is selected from the group consisting of an agent for treating brain-related disorders, a chemotherapeutic agent, and a gene-therapy agent.

63. The method of claim 46, wherein the at least one agent is delivered to the target location in bolus.

64. The method of claim 46, wherein the target location is in or close to a tissue in the subject, wherein the tissue has a pathological condition.

65. The method of claim 64, wherein the target location is the artery in or near brain, a tumor, or a tissue in need of gene-therapy.

66. The method of claim 46, wherein the subject is a mammal.

67. The method of claim 46, wherein the subject is a human.

68. A method for the localized delivery of an agent to a target location within a subject, comprising the steps of:
(1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated;
(2) incorporating the agent into the drug delivery system;
(3) occluding blood flow to the target location by inflating the balloon;
(4) delivering the agent in bolus to the target location; and
(5) deflating the balloon after a short period of time.

69. A method for the treatment of a pathological disorder in a subject, comprising the steps of:
(1) partially or completely arresting blood flow to a target tissue for a short period of time;
(2) delivering at least one therapeutic agent in bolus; and
(3) partially or completely restoring blood flow to the target tissue, wherein the target tissue has a pathological condition and the blood flow is arrested by occluding the artery to the target tissue, and wherein the at least one therapeutic agent is delivered into the target tissue or a location within the artery which is close to the target tissue.

70. The method of claim 69, wherein the steps of (1)-(3) are repeated at least once.

71. The method of claim 69, wherein the blood flow is arrested by inflating a balloon and restored by deflating the balloon.

72. The method of claim 71, wherein the balloon is inflated or deflated in about 1 second.

73. The method of claim 71, wherein a balloon drive controls the inflation and deflation of the balloon.

74. The method of claim 73, wherein a computerized device controls the balloon drive.

75. The method of claim 71, wherein the duration of the balloon inflation is about 10-50 seconds.

76. The method of claim 69, wherein the ventilation of the subject is increased.

77. The method of claim 69, wherein the at least one therapeutic agent is delivered using a steerable device.

78. The method of claim 77, wherein the steerable device comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated.

79. The method of claim 69, wherein the target tissue is brain, a tumor, or a tissue in need of gene-therapy.

80. The method of claim 69, wherein the subject is a mammal.

81. The method of claim 69, wherein the subject is a human.

82. A method for the treatment of a pathological disorder in a subject, comprising the steps of:
(1) providing a drug delivery system comprises a steerable device and a balloon drive, wherein the steerable device comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated;
(2) incorporating at least one therapeutic agent into the drug delivery system; and
(3) delivering the at least one therapeutic agent to a target location, wherein the target location is in or close to a target tissue in the subject, wherein the target tissue has a pathological condition.

83. The method of claim 82, wherein the micro-catheter is extended beyond the distal end of the balloon.

84. The method of claim 82, wherein the catheter is made of a material with high tensile strength.

85. The method of claim 84, wherein the high tensile strength material is selected from the group consisting of Teflon, nylon, polyurethane, and polyethylene.

86. The method of claim 82, wherein the catheter has a surface coating.

87. The method of claim 86, wherein the surface coating is hydrophilic.

88. The method of claim 82, wherein the balloon drive controls the inflation and deflation of the balloon.

89. The method of claim 82, further comprising a computerized device, wherein the computerized device controls the balloon drive.

90. The method of claim 89, wherein the primary parameters used by the computerized device to control the balloon drive are the frequency, duration, and volume of inflation/deflation.

91. The method of claim 82, wherein the balloon is inflated by a radio-opaque low viscosity fluid.

92. The method of claim 82, wherein the balloon can be inflated or deflated in about 1 second.

93. The method of claim 82, wherein the duration of the balloon inflation is about 10-50 seconds.

94. The method of claim 82, wherein the balloon inflation partially or completely blocks the blood flow to the target location.

95. The method of claim 82, wherein the balloon is rapidly inflated and deflated at least once.

96. The method of claim 82, wherein the ventilation of the subject is increased.

97. The method of claim 82, wherein the at least one therapeutic agent is selected from the group consisting of an agent for treating brain-related disorders, a chemotherapeutic agent, and a gene-therapy agent.

98. The method of claim 82, wherein the target location is the artery in or near brain, a tumor, or a tissue in need of gene-therapy.

99. The method of claim 82, wherein the therapeutic agent is delivered to the target location in bolus.

100. The method of claim 82, wherein the subject is a mammal.

101. The method of claim 82, wherein the subject is a human.

102. A method for the treatment of a pathological disorder in a subject, comprising the steps of:
(1) providing a drug delivery system comprises a catheter and a balloon drive, wherein the catheter comprises a proximal double-lumen assembly and a balloon, wherein the first lumen of the double-lumen assembly is a micro-catheter and the second lumen of the double-lumen assembly is a larger lumen for inflating and deflating the balloon, and wherein the balloon can be rapidly inflated and deflated;
(2) incorporating at least one therapeutic agent into the drug delivery system;
(3) occluding blood flow to the target location by inflating the balloon;
(4) delivering the at least one therapeutic agent to the target location; and
(5) deflating the balloon after a short period of time.

103. The method of claim 102, wherein the steps (3)-(5) are repeated at least once.
